(19) United States
(12) Patent Application Publication
(10) Pub. No.: US 2024/0050288 A1
(43) Pub. Date: Feb. 15, 2024

(54) DURABLE ABSORBENT PANT WITH IMPROVED SEAMING

(71) Applicant: The Procter & Gamble Company, Cincinnati, OH (US)

(72) Inventors: Ashley BASIUS, Cincinnati, OH (US); Yoel EYAL, Tel Aviv (IL); Eiro FUKUDA, Mason, OH (US); Jessica HEMMER, Cincinnati, OH (US); Cassandra SEPELLO, Loveland, OH (US); Madeleine Claire TEPE MISLEH, Cincinnati, OH (US); Ellen Kramer Der KRIKORIAN, Cincinnati, OH (US)

(21) Appl. No.: 18/447,363

(22) Filed: Aug. 10, 2023

Related U.S. Application Data
(60) Provisional application No. 63/397,523, filed on Aug. 12, 2022.

(51) Int. Cl. A61F 13/496; A61F 13/49; A61F 13/494

(52) U.S. Cl. CPC A61F 13/4963; A61F 13/49006; A61F 13/49426; A61F 2013/49084

(57) ABSTRACT

A durable absorbent pant is disclosed. The pant includes an outer pant structure and a gusset assembly disposed at least partially in a crotch portion. The gusset assembly includes an absorbent layer, a barrier layer beneath the absorbent layer, and an edge barrier overlying portions of a wearer-facing surface of the gusset assembly along a perimeter edge of the gusset assembly, and effectively continuing over and around the perimeter edge to an outward-facing surface of the gusset assembly. The edge barrier may be formed of a liquid impermeable edge sealing strip material joined to the barrier layer, or, the edge barrier may be formed of an extension of the barrier layer. The gusset assembly may be affixed within the crotch portion by various disclosed mechanisms.

### BACKGROUND

Currently, the market for durable (washable/reusable) absorbent underwear pants is growing. For purposes of managing menstrual discharge or unintended urinary discharge resulting from incontinence, increasing numbers of women are opting to use durable absorbent underwear in conjunction with, as a supplement to, or even as a substitute for, conventional disposable feminine hygiene pads, tampons, disposable incontinence pads and/or disposable absorbent underwear.

Several designs for durable absorbent underwear pants are currently in the market. The simplest form resembles a conventional pair of underwear briefs, with a crotch section or “gusset” including one or more added layers of absorbent fabric, underlaid by a liquid-impermeable barrier layer, included and sewn into the crotch portion of the underwear. To address and mitigate the potential for wicking of absorbed fluid laterally through the absorbent material outwardly to the leg opening edges where it may reach and soil outer clothing, several designs have included a peripheral edge barrier that wraps about one or more layer components of the gusset and in some designs, the outer pant fabric. To further eliminate potential fluid wicking and leakage pathways created by incorporation of stitching, several designs have replaced stitching/sewing with strips or tapes formed of heat-activated adhesive material which are typically sandwiched between respective materials to be joined, and then subjected to heat and pressure (e.g., via a hot iron or press) to affix and adhere layers together. This can help the manufacturer to avoid use of stitching, which may require puncturing and inserting thread through an absorbent gusset assembly and its barrier components, creating potential fluid wicking or leakage pathways.

The use of adhesive strips or tapes to join material layers in active regions of a garment, however, may have its own drawbacks. Even where the compound(s) constituting the adhesive is/are formulated to impart flexibility and elasticity to the adhesive material, an extended and continuous formation of the adhesive material and/or the penetration of the adhesive material into the interstitial spaces within fabric knit structures, with adhesion to the constituent fibers/yarns of the joined fabrics, has a binding effect, adding stiffness and reducing elastic stretchability of the materials along the regions joined by the adhesive. In areas in which flexibility and elastic stretchability is particularly desired for purposes of secure fit and wearer comfort, e.g., about leg openings, this is undesirable. Additionally, where such tapes are included proximate to leg opening edges, they add to material stack up present at the leg edges, adding caliper and creating a potential source of potential discomfort at the leg edges, and contributing to “panty lines” which may be visible through outer clothing, and deemed undesirable by the wearer. Consequently, room for improvement exists in structures and methods for assembling absorbent underwear that minimizes the potential for leakage of absorbed fluid while preserving material flexibility and stretchability.

### BRIEF DESCRIPTION OF THE DRAWINGS

FIG. 1 is a simplified depiction of an example of a brief pant, as it would appear laid out flat on a horizontal planar surface, front waist portion facing up.

FIG. 2 is a depiction of an example of the brief pant of FIG. 1, in an assembled but opened configuration wherein the front and rear waist portions have been separated at the hip portions or hip side seams, as it would appear laid out flat on a horizontal planar surface, wearer-facing surfaces facing up.

FIGS. 3A-3C are partial, schematic lateral cross section views of examples of absorbent gusset assemblies, through a side edge proximate to a leg opening.

FIGS. 4A and 4B are partial, schematic longitudinal cross section views of examples of pants including absorbent gusset assemblies, through a crotch portion and leg opening edge.

FIGS. 5, 6A-6C, 7A-7C, 8 and 9 are partial, schematic longitudinal cross section views of additional examples of pants including absorbent gusset assemblies, through a crotch portion and leg opening edge.

FIGS. 10A-10C are partial, schematic longitudinal cross section views of examples of pants including absorbent gusset assemblies, through an end of the gusset assembly.

FIG. 11 is a schematic vertical cross section, through a longitudinal center plane (as identified in FIG. 1) of one possible example of a pant with a gusset, oriented as it would be on a standing wearer.

FIG. 12A is a simplified depiction of an example of a brief pant, as it would appear laid out flat on a horizontal planar surface, front waist portion facing up.

FIG. 12B is a simplified depiction of an example of a shorts pant (or legged pant), as it would appear laid out flat on a horizontal planar surface, front waist portion facing up.

FIG. 13A is a plan view of an example of a crotch gusset laid out on a horizontal surface, wearer-facing surface facing the viewer.

FIG. 13B is a schematic vertical cross section of a portion of the gusset shown in FIG. 13A.

### DETAILED DESCRIPTION

### Definitions

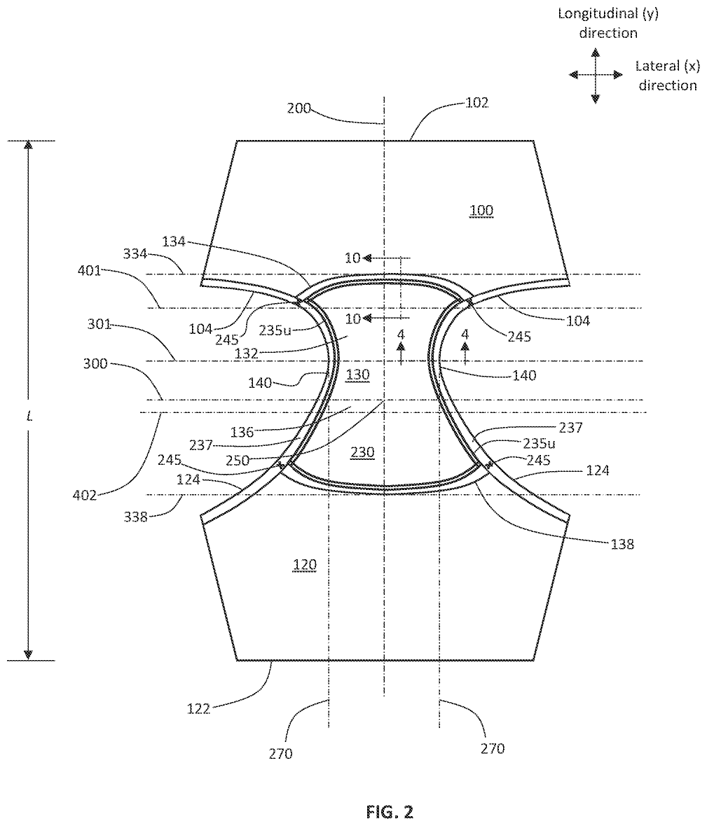

A “crotch gusset 230” is a structural component that includes at least two distinct layers including an absorbent layer and a liquid impermeable barrier layer, and bridges the front waist portion 100 and the rear waist portion 120. A crotch gusset has a “liquid impermeable barrier layer” if it exhibits z-Direction Leakage no greater than 0.1 ml of test fluid into a section of filter paper, in the Liquid Impermeability test described below. Referring to FIG. 2 by way of example, for purposes herein, crotch portion 130 is the portion of the pant, at a minimum, lying between crotch portion minimum front extent 401 and crotch portion minimum rear extent 402, longitudinally centered about crotch portion lateral axis 301 (which is drawn along the smallest width dimension measured between the crotch leg opening edges 140), plus 10 percent of the overall length L of the pant to the front and the rear of the crotch portion lateral axis 301. As suggested by FIG. 2, it may be desired that the crotch portion lateral axis 301 be disposed forward of the lateral axis 300 (which equally divides overall length L), rather than be co-located with lateral axis 300, for purposes of better fit about an adult female wearer's legs and lower torso. Thus, the respective boundaries between crotch portion 130 and front and rear waist portions 100, 120 for purposes herein are independent of the location(s) of any seams such as seams 134, 138 that may be present to join material(s) included in the crotch gusset 230 and material(s) included in the front and rear waist portions 100, 120.

With respect to a wearable garment such as an underwear pant, “durable” means that the garment is made predominantly of fabric that is knitted and/or woven from natural, semi-synthetic or synthetic fiber, thread or yarn, and may be normally laundered, or hand-washed and dried, for reuse/re-wear a plurality of times without substantial loss of original shape, structural integrity, absorbent function or other useful mechanical attributes that existed prior to laundering or washing.

As used herein, “fabric” means a web material that is knitted or woven of fibers, or threads or yarns of fibers. Herein, the term “fabric” does not include a film or a fibrous nonwoven web material.

As used herein, “film” means a skin- or membrane-like material that is cast, extruded or formed in place, from a molten thermoplastic material. Herein the term “film” does not include fibrous nonwoven web material or fabric.

With respect to a pant in an assembled but opened configuration, laid out flat on a horizontal planar surface, “longitudinal” refers to a direction generally perpendicular to a line tangent each of the left and right leg opening edges where they are closest the front waist edge. With respect to a pant in an assembled configuration, laid out flat on a horizontal planar surface, front waist portion facing up, “longitudinal” refers to a direction generally perpendicular to a line tangent each of the left and right leg opening edges where they are closest the front waist edge. “Length” refers to a dimension measured along the longitudinal direction. The longitudinal direction is deemed a “y-direction” in figures where indicated.

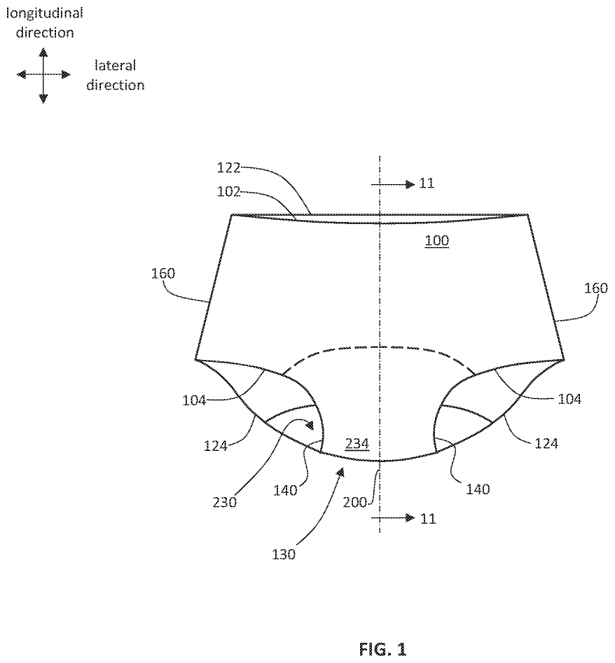

With respect to a pant in an assembled but opened configuration (e.g., as illustrated in FIG. 2), laid out flat on a horizontal planar surface, “lateral” refers to a direction generally parallel to a line tangent each of the left and right leg opening edges where they are closest the front waist edge. With respect to a pant in an assembled configuration (e.g., as illustrated in FIG. 1), laid out flat on a horizontal planar surface, front waist portion facing up, “lateral” refers to a direction generally parallel to a line tangent each of the left and right leg opening edges where they are closest the front waist edge. “Width” refers to a dimension measured along the lateral direction. The lateral direction is deemed an “x-direction” in figures where indicated.

With respect to a pant in an assembled but opened configuration (e.g., as illustrated in FIG. 2), laid out flat on a horizontal planar surface, the “z-direction” is the direction orthogonal to the longitudinal and lateral directions, i.e., the vertical direction relative the horizontal planar surface.

With respect to respective layer components in a crotch gusset of a pant in an assembled but opened configuration (e.g., as illustrated in FIG. 2), laid out flat on a horizontal planar surface, wearer-facing surfaces facing up, as between first and second layer components in the crotch portion, the terms “above,” “upper,” “superadjacent,” “below,” “lower,” “subjacent” and/or “beneath” describe the components' disposition along the z-direction relative each other. Thus, for example, referring to FIG. 4A, a wearer-facing layer 231 is disposed “above” an outward-facing layer 234, and conversely, the outward-facing layer 234 is disposed “below” the wearer-facing layer 231 and/or absorbent layer 232. “Superadjacent” and “subjacent” with respect to two layer components, mean further, that the two layer components are disposed in direct surface-to-surface contact with each other.

A yarn, thread, fiber, filament, web, film or fabric material, or a laminate or composite of any of these, is considered to be “elastically extensible” for purposes herein if, when a tensile force no greater than 50 gf/mm (gf per mm of sample width, where sample width is measured perpendicular to the stretch direction) is applied to the subject material along a stretch direction, the material may be extended along the direction to an elongated dimension of at least 130% of its original relaxed dimension (i.e., can extend at least 30%), without rupture or breakage which substantially damages the subject material; and when the force is removed from the subject material, the material retracts along the stretch direction to recover at least 40% of such elongation. To illustrate, if a section of fabric having an original relaxed length of 100 mm and a sample width of 40 mm can be elongated by tensile force of 2000 gf (50 gf/mm) in a direction along its length to 130 mm length without substantial damage, and will retract upon removal of the force to a length no greater than 118 mm (130 mm−118 mm=12 mm=40% of 30 mm), it is “elastically extensible” as defined herein. “Elongation,” used herein to quantify and express an amount of strain imparted to an elastically extensible material in a stretch direction, means: {[(strained length of the strand)−(length of the strand prior to straining)]/(length of the strand prior to straining)}, ×100%. For a monolithic material such as a cast or extruded film material, “elastomeric” means “elastically extensible” as defined herein.

With respect to two opposing surfaces of a layer component of a pant, “wearer-facing” refers to the surface that faces the wearer's skin when the pant is worn normally; and “outward-facing” refers to the surface that faces away from the wearer's skin. With respect to two distinct layered components of a pant, the “wearer-facing” component is the component that is disposed closest the wearer's skin when the pant is worn normally; and the “outward-facing” component is the component that is disposed farthest from the wearer's skin.

For purposes herein, “pant” includes any garment adapted for wear about the human lower torso, including a front waist portion and a rear waist portion that join about the wearer's hips and beneath the wearer's crotch, to form a garment having a waist opening and a pair of leg openings. Herein, the term “pant” encompasses (but is not limited to) a garment defined herein as a “brief pant”; a garment defined herein as a “legged pant”, and any other garment whether adapted for use as underwear or outerwear, having such features.

For purposes herein, unless otherwise specified, with respect to the proportionate content of a component material in a combination or structure, “predominant” means the component constitutes the majority of the weight of the combination or structure.

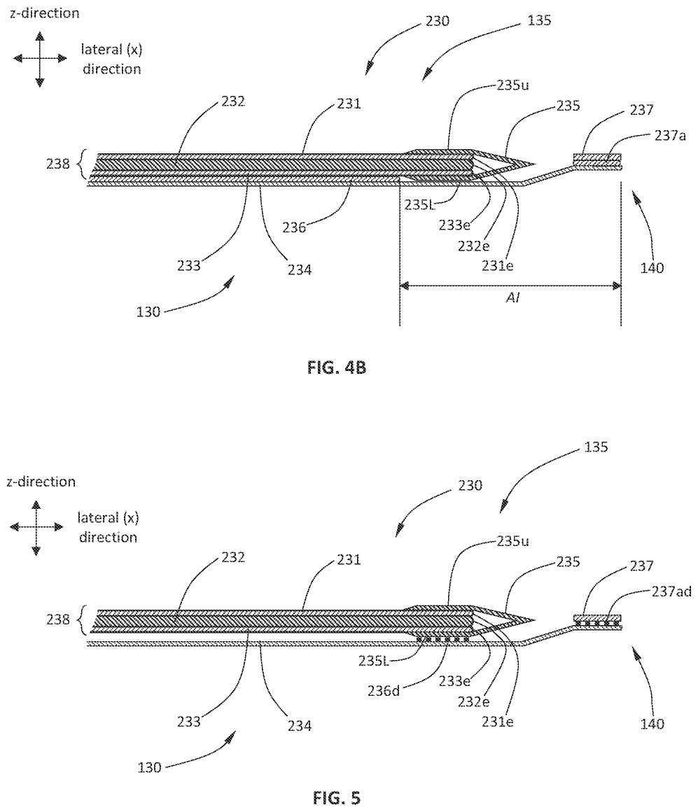

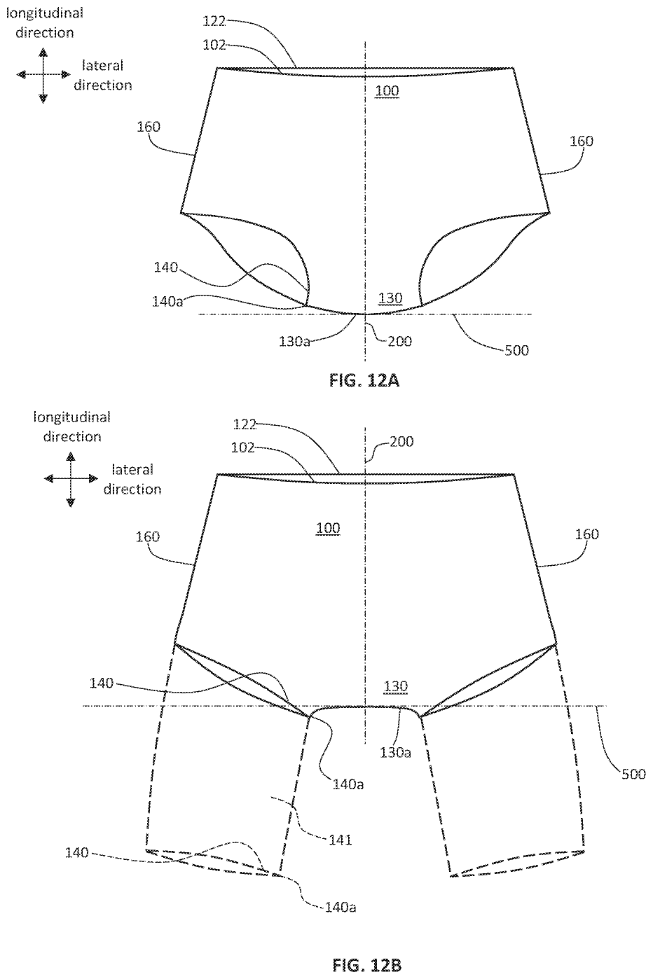

For purposes herein, a “brief pant” is distinguished from a “legged pant” by the configuration of the inside leg edges, resulting from the manner in which the component materials are shaped, sized, proportioned and seamed or otherwise affixed together. FIG. 12A depicts an example of a brief pant and FIG. 12B depicts an example of a legged pant in alternative forms. When the garment in its assembled condition is laid out flat on a horizontal planar surface, front waist portion facing up, and a lateral crotch tangent line 500 is drawn perpendicularly to longitudinal axis 200 and tangent to the point at which the crotch portion lower profile 130a intersects the longitudinal axis 200,
for a brief pant, the lowermost points 140a along the inside leg opening edges 140 are disposed along or above the crotch tangent line 500 (i.e., toward the front waist edge 102) (see FIG. 12A); and
for a legged pant, the lowermost points 140a along the inside leg opening edges 140 are disposed below the crotch tangent line 500 (i.e., away from the front waist edge 102) (see FIG. 5B).

As reflected in FIG. 12B, for purposes herein, a legged pant may have leg portions 141 of any length, wherein the lowermost points 140a of inside leg opening edges 140 are disposed below the crotch tangent line 500. Underwear shorts pants such as “boy short” styles will generally have shorter leg portions as suggested in the solid-line portions of FIG. 12B, while other legged pants may have leg portions of varying lengths, as suggested by the dashed-line portions of FIG. 12B.

### DESCRIPTION

One problem presented in designing an absorbent system to be used to contain and absorb discharged menstrual fluid and unintended (incontinent) small discharges of urine by a woman results from adhering flow. As a result of relatively low velocity of discharges, female anatomical features, and typical surface chemistry of human skin (which can cause skin surfaces to attract and be relatively wettable by aqueous solutions such as menstrual fluid and urine), adhering flow has always been a phenomenon associated with menstruation and low to moderate adult female incontinence. If features of a chosen containment/absorbency system do not reliably hold absorbent materials against the wearer's skin to intercept adhering flow, leakage can result.

It is believed, however, that a durable adult absorbent brief pant (i.e., a pant with a crotch portion including an absorbent structure and a liquid impermeable barrier layer to the outside thereof), that reliably protects against leakage of adhering flow through a variety of body movements and positions and over a reasonable duration of wear/use has not been marketed to date. Currently marketed pant products do not effectively hold included absorbent structures against the body through various body movements, over a reasonable duration of wear/use. It is believed that the garment industry to date has failed to recognize that suitably oriented elastic stretch is important for maintaining proximity and/or contact of the absorbent structure with the wearer's body contours and skin surfaces through various body positions and movements, and that a structure having a combination of absorbent capacity suitable for providing protection against leakage resulting from menstrual flow or light to moderate incontinence, while having suitable directional elastic stretch so as to maintain effective contact of the structure with the wearer's body, may be designed. The human body and particularly the female lower torso and crotch region have geometrically non-ruled contoured surfaces. Fabrics or web materials that do not exhibit an effective amount of elastic stretch capability along an effective direction will not effectively conform closely to (i.e., remain in contact with) the substantial majorities or entireties of these surfaces through ordinary body positions and movements. Increasing the amount of elastic stretch capability imparted to crotch gusset increases the variety of body shapes and movements to which the gusset can conform in use. In connection therewith, reducing the longitudinal tensile modulus (longitudinal tensile force in the material resulting from longitudinal stretching) tends also to increase body conformity and enhance comfort.

For underwear, many women prefer brief pants rather than legged pants such as “boy shorts” styles for ordinary daily wear. This is due to issues of comfort; unlike a legged, shorts-type pant, a brief pant ordinarily will not ride up and bunch about the legs from changes of body position, and thereby be a source of unwanted concentration of material bulk, tightness about the legs or other discomfort under outer clothing. Further, due to the manner in which their leg edges tend to cause the pant to fit through the crotch region of the body, brief pants having suitable elastic stretch characteristics may be preferred for maintaining a close fit about the female genital/urethra area, for purposes of protecting against leakage of menstrual fluid or unintended discharges of urine.

It has been learned that a durable absorbent pant may be designed that more reliably and comfortably holds an absorbent structure against/in contact with a wearer's body through the crotch region, in a position to better intercept adhering flow, capture the discharged liquid in the absorbent material before it can escape, and permit expansion of the absorbent components, initially and over a reasonable duration of wear/use of the pad. In order for a fabric or web of material to conform to a non-ruled surface, it must be capable of stretching along at least one direction. It has been determined that the direction of stretch that most effectively enables a fabric or web of a pant garment to effectively conform to the contours of the female lower torso and crotch region is, substantially, the longitudinal direction.

Referring to FIGS. 1 and 2 as an illustrative but non-limiting example, a pant may include a front waist portion 100, a rear waist portion 120 and a crotch portion 130 bridging the front and rear waist portions. Front waist portion 100 has a front waist edge 102, and left and right front leg opening edges 104. Rear waist portion 120 has a rear waist edge 122, and left and right rear leg opening edges 124. Crotch portion 130 has left and right crotch leg opening edges 140. Crotch portion 130 may include the entirety or a portion of a crotch gusset 230 that may include several layer components that will be described further below. (For purposes herein, the “crotch portion 130” is a portion of a pant identified as described herein, and its forward and rearward extents are independent of specific components or structures.) Herein, a “gusset” 230 is a structural component that includes at least two distinct layers including an absorbent layer and a liquid impermeable barrier layer, and is disposed in the crotch portion and may bridge the front waist portion 100 and the rear waist portion 120. A gusset has a “liquid impermeable barrier layer” if it exhibits z-Direction Leakage no greater than 0.1 ml of test fluid into a section of filter paper, in the Liquid Impermeability test described below. Referring to FIG. 2 by way of example, for purposes herein, crotch portion 130 is the portion of the pant, at a minimum, lying between crotch portion minimum front extent 401 and crotch portion minimum rear extent 402. Crotch portion 130 is longitudinally centered about crotch portion lateral axis 301 (which is drawn along the smallest width dimension measured between the crotch leg opening edges 140), and includes 10 percent of the overall length L of the pant to the front and the rear of the crotch portion lateral axis 301. As suggested by FIG. 2, it may be desired that the crotch portion lateral axis 301 be disposed forward of the lateral axis 300 (which equally divides overall length L), rather than be co-located with lateral axis 300, for purposes of better fit about an adult female wearer's legs and lower torso. Thus, the respective boundaries 401, 402 between crotch portion 130 and front and rear waist portions 100, 120 for purposes herein, are independent of the location(s) of any seams such as seams 134, 138 that may be present to join material(s) included in the crotch gusset 230 and material(s) included in the front and rear waist portions 100, 120. Material forming the front and rear waist portions 100, 120 may be integral and continuous across, or may be joined at seams, at or proximate hip portions 160, to complete a pant structure having a waist opening and a pair of leg openings.

Material(s) forming one or both of forward and rearward portions 132, 136 of crotch portion 130 may be integral and continuous with material(s) forming front and rear waist portions 100, 120, or alternatively, one or both of forward and rearward portions 132, 136 of crotch portion 130 and/or crotch gusset 230 may be substantially formed of one or more sections or layers of material that are distinct from material(s) substantially forming one or both of front and rear waist portions 100, 120, and crotch gusset 230 may be joined to front and rear waist portions 100, 120 at one or both of forward seam 134 and rearward seam 138. In illustrative but non-limiting examples suggested in FIGS. 1, 2 and 10A-10C, front waist portion 100, an outward-facing layer 234 of crotch portion 130, and rear waist portion 120, may all be formed partially or entirely of a first single, continuous section of material, e.g., the fabric from which the main body of the pant is formed. As reflected in the examples shown in FIGS. 4A, 4B, 5, 6A-6C, 7A-7C, 8, 9 and 10A-10C, one or more additional layers of material 231, 232, 233 may be disposed over the wearer-facing side of outward-facing layer 234 in crotch portion 130 as shown, and be connected or affixed directly or indirectly to layer 234 via, e.g., stitching/sewing, adhesive bonding, thermal bonding (fusing or welding) or other suitable attachment/joining mechanism (hereinafter, attachment mechanism) at, e.g., forward and rearward gusset seams 134, 138 and/or along the crotch leg opening edges 140.

In other non-limiting examples (not specifically shown), the sections of materials respectively forming front waist portion 100, rear waist portion 120 and crotch portion 130 may be entirely separate and distinct, and joined via any suitable attachment mechanism at, e.g., forward and rearward seams 134, 138. This configuration may be preferred in some circumstances because it may provide the designer with greater flexibility in selection of the respective materials for the waist and crotch portions with respect to appearance, feel, weight, breathability, elongation, stretch characteristics and cost. The elongation and stretch characteristics described below would be applicable to the one, or more layers of material in combination, present in crotch portion 130 and/or crotch gusset 230.

Crotch Portion/Crotch Gusset

Gusset Components

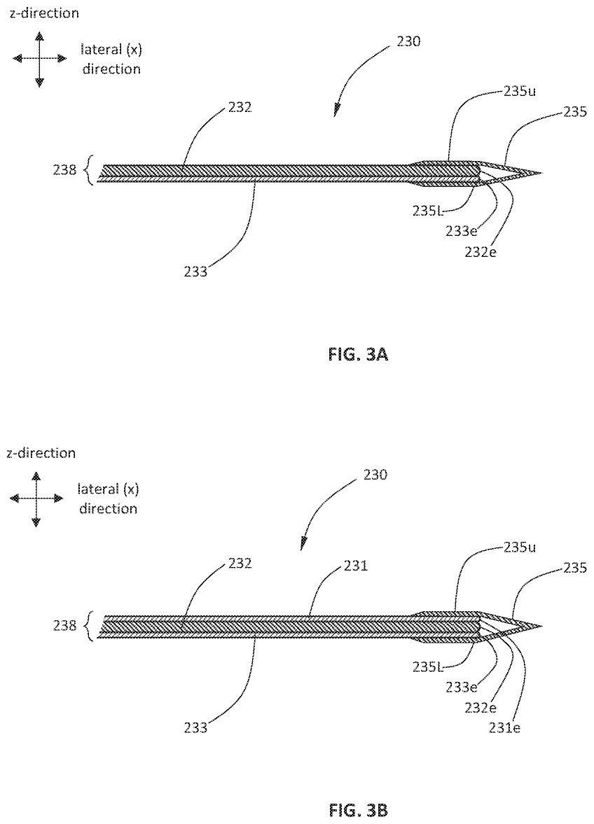

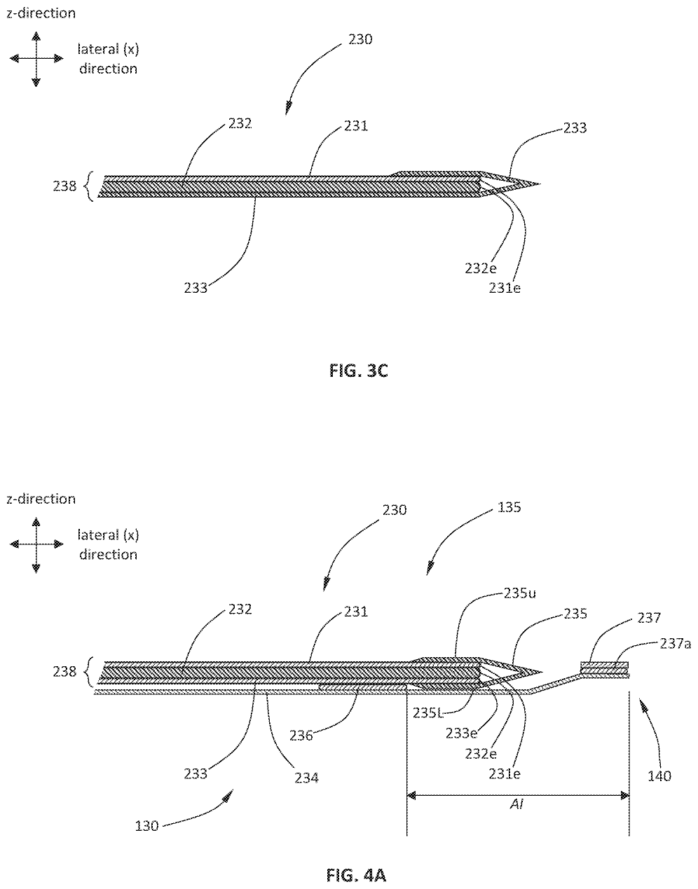

Referring to FIGS. 3A, 3B and 3C, a crotch gusset 230 includes components of an absorbent assembly 238 which include, at a minimum, an absorbent layer 232 and a barrier layer 233. The absorbent layer 232 may be one or more layers of suitable fabric, selected for desired absorbency and stretch characteristics. Absorbent layer 232 will be underlaid by a barrier layer 233, which may be a suitable polymeric film, or alternatively, a fabric that is effectively resistant to passage of liquid therethrough under ordinary conditions of wear of garments of the type described herein. Barrier layer 233 serves to prevent liquids absorbed by the absorbent layer 232 from passing by wicking or capillary action out to an outward-facing layer of the pant, and from there out to outer clothing, bedclothes, etc.

In some examples (not specifically shown), absorbent layer 232 may include or be formed of two, three or more distinct sublayers of material disposed superadjacent/subjacent one another. The respective sublayers may have different sizes, dimensions and/or surface areas along the x- and y-directions, and may be formed of or include respectively differing materials. In some examples, one or more of the sublayers may be formed of or include a nonwoven web material. In such examples, for reasons explained herein, it may be preferred that such sublayers be constituted and manufactured so as to possess elastic extensibility, or alternatively, be arranged within the gusset so as to be unbonded/unattached to superadjacent or subjacent layers (e.g., elastically extensible layers), over a majority of the overlapping surface area therebetween, to avoid impeding elastic stretch of such superadjacent/subjacent layers.

Barrier layer 233 may be formed of or include a suitable polymeric film. The polymer component(s) of the film may be selected so as to impart one or both of effective liquid impermeability and elastic extensibility thereto. In some examples, the film may be adapted to be liquid impermeable under ordinary conditions of use contemplated herein, but be effectively gas and/or vapor permeable (i.e., “breathable”), so as to allow water vapor to more easily escape the garment, and thereby improve comfort and reduce chances for overhydration of the wearer's skin (which can cause or contribute to undesirable growth of microorganisms, skin irritation, rash, etc.). In some examples, it may be preferred that barrier layer 233 be unattached to overlying absorbent layer(s) in middle/central regions of the gusset between peripheral edge seams, to reduce binding, stiffening and elasticity-reducing effects of bonding or affixing superadjacent/subjacent layers.

In some examples crotch gusset 230 may include an additional, overlying wearer-facing layer 231. Wearer-facing layer 231 may be formed of a suitable fabric that has a comfortable feel against the skin while being configured to function to conduct liquids down to the absorbent layer(s) 232 following a discharge, and subsequently, to isolate the absorbed fluids in the absorbent layer from the wearer's skin, to help impart a relatively dry feel to the pant for the wearer, even after a discharge.

In most designs it may be preferred to provide edge sealing of the absorbent layer 232 and wearer-facing layer 231 (if included), about the perimeters of these layers, e.g., about a portion or the entirety of the perimeter of the gusset 230, or at least at the perimeter edges of the gusset proximate the leg opening edges 140 in the crotch portion 130 of the pant. Since these layers may be formed of fabrics configured to accept and transfer liquid via wicking or capillary action, it may be desired to block liquid movement out the perimeter edges of these layers, which can result in leakage and soiling of outward-facing layer 234 of the crotch portion, outer clothing, bedclothes, etc.

Referring to FIGS. 3A and 3B, in some examples, edge sealing may be provided by including an edge sealing strip 235. Edge sealing strip may be formed of a film or fabric that is suitably waterproof or effectively resistant to passage of liquid therethrough, under conditions of use contemplated herein. Edge sealing strip 235 is preferably sealingly adhered to barrier layer 233 so as to be effectively unitary therewith, and wraps up and around the perimeter edges of the absorbent layer(s) 232 and wearer-facing layer 231 (if included), and is affixed to the wearer-facing surface of the uppermost layer proximate the perimeter thereof. So configured, edge sealing strip can be effective to block passage of liquid that may wick to the perimeter edges of these layers, out therefrom. Edge sealing strip 235 may be formed of an inherently tacky material having sufficient adhesive properties to enable it to adhere to barrier layer 233 and wearer-facing layer 231 with or without application of heat and pressure, or alternatively, edge sealing strip 235 may be adhered to these component via a suitable adhesive. For purposes of retaining any inherent elastic stretchability of individual layers 231, 232, 233 in the assembly, it may be desired that edge sealing strip 235 be formed of an elastomeric material. In some examples, edge sealing strip 235 may be formed of a substantially liquid impermeable polymeric film having partially or predominantly a silicone (polysiloxane) component, which may be preferred for its relative softness, pliability, elasticity/elastic extensibility, durability and resilience.

In other examples such as suggested in FIG. 3C, edge sealing may be provided by an integral, continuous extension of barrier layer 233, providing a perimeter margin portion thereof of a size sufficient to wrap up and around the perimeter edges of the absorbent layer(s) 232 and wearer-facing layer 231 (if included), and be affixed via adhesive or other suitable mechanism to the wearer-facing surface of the uppermost layer proximate the perimeter thereof. So configured, such wrapping margin portion(s) of barrier layer 233 can be effective to block passage of liquid that may wick to the perimeter edges of the absorbent layer(s) 232 and wearer-facing layer 231 (if included), out therefrom.

Caliper

For purposes of minimized bulk, it may be desired that the combination of layered materials present in the crotch portion 130 and crotch gusset 230 be selected and configured so has to have a central z-direction Caliper not exceeding about 6 mm, more preferably not exceeding about 5.5 mm, and even more preferably not exceeding about 5 mm. (Herein, the “central z-direction Caliper” or “central Caliper” is measured at a location in the crotch portion 130 corresponding with the intersection 250 of the longitudinal 200 and lateral 300 axes of the pant (shown in FIG. 2), when opened and laid out flat on a horizontal planar surface.) Based on disclosure herein and teachings available in the art concerning textiles, persons of ordinary skill in the art will be equipped to select a combination of suitable materials to create a structure in the crotch portion 130 having such Caliper, along with other features and attributes described herein.

Absorption Capacity

In order for a durable absorbent pant to provide suitable absorbency and protection against leakage of menstrual fluid or unintended discharges of urine over a reasonable duration of wear, for a woman experiencing menstruation or light to moderate incontinence, while balancing concerns for limiting caliper/bulkiness of the crotch portion while limiting the required relative planar size/surface area of an absorbent portion, it may be desired that a combination of material forming the structure within the crotch portion 130 of the pant have an Area Absorption Capacity from 0.1 ml/cm² to 0.4 ml/cm², and a Volume Absorption Capacity from 0.4 ml/cm³ to 1.0 ml/cm³, measured according to the Absorption Capacity Measurement method set forth below. Based on disclosure herein and teachings available in the art concerning textiles, persons of ordinary skill in the art will be equipped to select a combination of suitable materials to create a structure in the crotch portion 130 having such absorption capacity.

Elastic Stretch Attributes

Through experimentation it has been learned that imparting a pant with appropriate directional elastic stretch characteristics within the crotch portion 130 are important to provide a pant with a crotch portion that is both comfortably and securely held against the wearer's body surfaces, in position to intercept and absorb an adhering flow of menstrual fluid or urine, and thereby prevent leakage from the pant, through normal ranges of body positions and movements. It has been learned that the combination of materials present in the crotch portion 130 preferably should exhibit a maximum Longitudinal Elongation of 25 percent to 100 percent, measured according to the Maximum Elongation Measurement Method set forth below. Alternatively, or additionally, it may be desired that the combination of materials present in the crotch portion 130 exhibit a Longitudinal Tensile Modulus, measured according to the Longitudinal Tensile Modulus Method set forth below (reflecting the presence of one or more materials that impart elasticity) of 10 gf/mm to 100 gf/mm. Based on disclosure herein and teachings available in the art concerning textiles, persons of ordinary skill in the art will be equipped to select a combination of suitable materials to create a structure in the crotch portion 130 having such longitudinal elongation and tensile modulus properties.

Materials Selection

Generally, the front and rear waist portions 100, 120 of suitable examples of a pant may be formed of any fabric material or combination of fabric and other materials known and used as components of underwear, swimwear or athletic/active wear, exhibiting suitable attributes that may include, depending upon the location of the fabric within the structure, pleasing feel against the skin (softness and/or low-friction/smooth/silky feel), low caliper/bulk, elongation capability, elastic extensibility, absorbency, wicking ability, breathability, etc. In one example, the front and rear waist portions 100, 120, and the outward-facing layer 234 of the crotch gusset 230, may include or be formed of a knitted stretch fabric, in some examples, a combination of one or more of nylon, polyester, cotton and elastane fibers (e.g., LYCRA SPANDEX (a product of The Lycra Company, Wilmington, DE)).

Additional layer components of crotch portion 130 and/or crotch gusset 230, as herein described requiring absorbency and longitudinal elastic stretch attributes, may include a combination of several materials selected to impart the structure with the desired attributes.

Fabric Structure

Most durable fabrics exhibit anisotropic elongation capabilities.

Woven fabrics, formed by weaving, are formed of two groups (warp group and weft group) of interlaced constituent yarns or threads, the yarns or threads within each group being substantially parallel to each other, and substantially perpendicular to the yarns or threads in the other group, along the plane of the fabric. Unless the constituent yarns or threads are themselves formed of extensible material, woven materials have relatively low elongation capabilities along the warp and weft directions, and have relatively higher elongation capabilities along the two bias directions approximately 45 degrees from the warp and weft directions. Consequently, where it is desired for particular reasons that a layer component of the crotch portion 130 and/or crotch gusset 230 be a woven material, it may be desired that the material be oriented within the crotch portion such that one of the warp and weft directions is oriented from approximately 30 degrees to approximately 60 degrees, preferably from approximately 38 degrees to approximately 52 degrees, and more preferably approximately 45 degrees from the longitudinal direction of the pant, so as to provide maximum available longitudinal elongation capability for the woven layer.

However, when stretched along the bias direction, woven fabrics typically exhibit a substantial Poisson effect contraction along the trans-stretch direction (90 degrees from the stretch direction). When such a fabric is included in the crotch portion 130 with its bias oriented approximately along the longitudinal direction of the pant, the Poisson contraction effect may cause the crotch portion to laterally narrow, which may be deemed undesirable when effective coverage of the wearer's body through the crotch region is desired.

The constituent yarns or threads of knitted fabrics, by contrast, do not follow straight paths along the plane of the fabric, and are neither parallel nor perpendicular to each other. Rather, each constituent yarn or thread of a knitted fabric follows a looping path along successive rows, interlooping with one or more constituent yarns or threads in adjacent rows. As a consequence, knitted fabrics exhibit relatively greater elongation capability along all directions as compared with woven fabrics, even where the constituent yarns or threads themselves are not extensible. For this reason, unless a woven fabric is desired for a particular reason, it may be preferred that a knitted fabric be used to form any one or more, or all, of the fabric layers present in the crotch portion 130 and crotch gusset 230 of the pant.

Even so, most types of knitted fabrics have elongation capabilities that are anisotropic along the plane of the fabric, having a first direction of greatest elongation capability and a second direction, perpendicular to the first direction, of least elongation capability. Accordingly, when knitted fabric is selected and used to form one or more layers present in the crotch portion 130 and crotch gusset 230 of the pant, it may be desired that the fabric(s) forming any, some or all of the layers be oriented such that their directions of greatest elongation capability are at least approximately parallel with the longitudinal direction of the pant.

In some circumstances it may be desired that a knitted fabric selected to form a layer be a rib knit type. Rib knitted fabrics exhibit relatively high elongation capability along a direction parallel to the knit rows (perpendicular to the “ribs”), with relatively low Poisson contraction effect along the trans-stretch direction. Thus, in some circumstances, it may be desired that one or more layers present in the crotch portion 130 and crotch gusset 230 of the pant be formed of a rib knit fabric, with the “ribs” oriented substantially along the lateral direction of the pant.

In some circumstances it may be desired that the absorbent layer 232 be formed of either woven or knitted terrycloth, for purposes of increasing aggregate fiber surface area and capillarity per unit fabric surface area, and thereby, providing increased absorbency to the absorbent layer 232, while still providing a durable fabric, in contrast to a nonwoven batt or matt of fibers. In conjunction therewith or as an alternative, and for purposes of enhancing absorbency, it may be desired that constituent fibers of the yarn(s) or thread(s) from which the absorbent layer fabric material is knitted or woven be in the form of microfibers (i.e., fibers having an average denier of one (1) or less). It may be further desired that the constituent fibers be split microfibers. Yarns or threads formed of microfibers, particularly split microfibers, provide relatively greater fiber surface area per unit yarn/thread denier. When the fiber surfaces are hydrophilic, this imparts relatively greater absorbency to the fabric.

Fabrics Constituent Yarn/Thread Compositions

As contemplated herein, wearer-facing 231 and absorbent 232 layers present in the crotch portion and crotch gusset of the pant are expected to be exposed to discharges of urine and/or menstrual fluid, and are expected to receive, absorb and retain these fluids for a reasonable duration of wear time, preferably while leaving the wearer-facing surfaces as dry-feeling as possible. It may be desired that a wearer-facing layer 231 have a soft feel against the skin.

Accordingly, it may be desired that the wearer-facing layer be formed of a material that has a soft feel and has suitable wicking attributes so as to efficiently conduct discharged liquid to an absorbent layer beneath, while having minimized tendency to retain liquid and thereby have a wet feel for the wearer. Thus, it may be desired that a wearer-facing layer 231 not include a predominant proportion of fibers typically used as absorbers, e.g., cotton or other plant fibers, or rayon fibers. (For purposes herein, the term “rayon” includes rayon, viscose, lyocell, and any other fibers spun from reconstituted/regenerated cellulose.) Preferably, the wearer-facing layer will not include any proportion of such fibers greater than 10 percent by weight of the fabric of the layer 234, and preferably, will include no substantial proportion of such fibers. Suitable materials may include polypropylene, polyesters and polyamides (e.g., nylon). Examples of these materials, when used to spin fiber components and/or when having received suitable hydrophilizing treatment, impart the spun fibers with suitable hydrophilic surface attributes (enhancing wicking), with relatively low individual fiber texture (reducing porosity and capillarity, and therefore, absorbency of the fabric). Additional materials may be incorporated in yarn or thread components for purposes of enhancing skin feel (e.g., enhancing a slick or silky feel against the skin) and/or further affecting hydrophilicity and/or reducing absorption tendencies. In some examples, polypropylene and/or polyethylene fiber components may be included for these purposes. In some examples, resins from which constituent fibers are spun may include additives to the primary polymer components, incorporated for enhancing skin feel, adjusting hydrophilicity, reducing absorbency, etc. In some examples, polyester or nylon component resins may include an additive comprising linolenic acid, to the extent of and as described in US 2017/0369698, for purposes of enhancing elongation and skin feel attributes, while reducing material usage and cost.

Although commercially processed cotton or other commercially processed plant fiber, or semi-synthetic, cellulose-derived materials such as rayon may be considered for inclusion as component material for the absorbent layer for their inherent absorbency attributes, some of these materials may have a tendency, undesirably, to retain constituents of menstrual fluid or urine following laundering. Accordingly, one or more of polyester, polyamide and/or combinations thereof may be preferred as component resins or even the main/predominant component resins from which fiber components of yarn or thread components of fabrics for the absorbent layer 232 are formed. The constituent threads or yarns of the absorbent layer may be knitted in a manner that imparts a relatively lofty and/or densely fibrous network having a degree of void volume and capillarity making it suitable for absorbing fluid. In some examples, the knit may be a knitted terrycloth, or even a knitted/sheared terrycloth. The constituent threads or yarns may be or may include microfibers, for increased fiber surface area per unit x-y surface area or volume of the absorbent assembly.

For purposes of imparting elastic extensibility to layers present in the crotch region (particularly longitudinal stretch), it may be desired that yarn or thread components of one or more of the fabrics present include elastically extensible fibers, yarns or threads. In some examples, elastically extensible fibers, yarns or threads may be formed of or include elastane or spandex (such as LYCRA, currently available from The Lycra Company, Wilmington, Delaware), which are particularly elastically extensible and durable through a plurality of launderings, as compared to other elastic materials used to elasticize fabrics.

In other examples, one or more elastically extensible polymer film layers distinct from the fabric layer(s) in the crotch portion 130 may be included to impart elastic extensibility to the structure in the crotch portion as a whole.

An elastic polymer film layer may be formed of any suitable elastic polymer material. In some examples, an elastically extensible film layer may be formed by extrusion or other application of film resin in molten or semi-molten form onto a layer component fabric, whereby the molten resin partially penetrates the fabric and upon cooling forms a film that is partially mechanically enmeshed in and/or made integral with the fabric.

Antimicrobial Agents

For purposes of hindering growth of microorganisms supported by components of absorbed urine or menstrual fluid, which may cause odor, it may be desired to include one or more antimicrobial agents in or among the materials present in the crotch portion 130. Any such antimicrobial agents are preferably included in a form adapted to remain in place and continue to be effective following a plurality of launderings of the pant. In some examples, an antimicrobial agent may include particles or fibers including a metal, metal alloy or metallic compound that includes one or more of copper, silver, zinc, aluminum or combinations thereof. In other examples an antimicrobial agent may include particles or fibers including carbon or a composition or compound including carbon. One or more of these materials may be included as additives to resins from which constituent fibers are spun, or may be included in compositions that are topically applied to constituent yarns, threads or fabrics following manufacture thereof. Such antimicrobial agents are preferably included in material(s) forming the absorbent layer 232 and/or the wearer-facing layer 231.

Barrier Layer

Where urine or menstrual fluid is absorbed by a fabric layer in the crotch portion 130 and crotch gusset 230, it may be desirable to include a barrier layer, e.g., barrier layer 233 (see, e.g., FIGS. 3A-3C) to prevent the absorbed fluid from passing from the absorbent layer to an outward-facing layer 234 or even to outer clothing. In some examples, a barrier layer 233 may be formed of or include a suitable liquid impermeable polymer film. In some examples, the film composition may be selected to have elastic extensibility, providing or complementing suitable elastic stretch attributes to the combination of layers present in the crotch portion 130 and/or crotch gusset 230. In some examples, a barrier layer 233 may be formed of or include a film formed in whole or in part of a polyurethane- or polyester-based resin. In some examples, a barrier layer 233 may be formed of or include a film that is formed by extrusion or other application of thermoplastic film resin in molten or semi-molten form directly onto the outward-facing surface of an overlying layer, such as absorbent layer 232, such that the film resin, while still molten, partially penetrates the fabric and thereby forms a liquid impermeable film that is partially mechanically enmeshed in and/or made integral with the fabric of the overlying layer. This also has the effects of consolidating these layers, which can reduce caliper in the crotch portion 130, and reducing or preventing wrinkling or bunching of the absorbent layer 232 upon elastic contraction. In a particular example, a polyurethane or polyester film may be formed by extrusion or other application of molten thermoplastic resin directly onto an outward-facing surface of a fabric that serves as or forms a component of absorbent layer 232. If the barrier layer 233 is separately formed, however, it may be desired to affixed it to the overlying layer, e.g. absorbent layer 232, via a suitable bonding mechanism, e.g., an adhesive disposed between the barrier layer and the overlying layer. A configuration that allows the barrier layer and the overlying layer to separate, so as to result in a void space between them in some circumstances, may be deemed undesirable because this may allow fluid to pool in the void space and thereby be uncontrolled and/or unmanaged by an absorbent structure, creating added risk of leakage. On the other hand, if the barrier layer 233 is not unified with an overlying fabric layer between peripheral regions of the gusset, the gusset may have enhanced pliability and stretchability since there is no binding effect resulting from integration of these layers. This latter construction may be preferred in circumstances in which, for example, management of only light menstruation or light incontinence is required, or alternatively, where the design of the garment otherwise has features that minimize the likelihood of void space between the barrier layer and the overlying absorbent layer(s).

The material selected for the barrier layer 233 may also be vapor permeable or “breathable” in that it can permit gas or water vapor to pass therethrough, while still being effectively liquid impermeable under ordinary conditions of the use contemplated herein, via a combination of having a porous structure for vapor permeability, but sufficiently small pore sizes and surfaces having low wettability (e.g. hydrophobic surfaces), for liquid impermeability. Various liquid impermeable, vapor permeable films and other materials are known and used in fields including personal hygiene and wound dressing applications. A liquid impermeable but vapor permeable barrier layer may be preferred in some circumstances for purposes of venting water vapor to improve wearer comfort and/or help avoid overhydration of the wearer's skin.

It some circumstances including those described herein (wherein, for example, the barrier layer forms a part of an edge sealing structure), it may be desired that the material(s) selected to form the barrier layer 233 be, or be processed to have, hydrophobic surfaces.

Gusset Seaming to Outer Pant Structure

The layers forming the gusset 230, e.g., wearer-facing layer 231, absorbent layer 232 and barrier layer 233, may be joined together to form an assembly, by any suitable mechanism. In some examples, these layers may be joined about their perimeters by a suitable adhesive interposed therebetween (not specifically shown). In some examples, these layers may be joined about their perimeters by stitching. If stitching is included for this purpose, it may be desired that the stitching not penetrate barrier layer 233 or an edge sealing structure such as edge sealing strip 235—to avoid creating liquid leakage pathways.

The following objectives are contemplated herein: Particularly about and proximate the leg opening edges 140 in the crotch portion 130 of the pant, it is deemed desirable to retain, as much as possible, elasticity and extensibility of the pant structure; to minimize material stack-up that may cause discomfort or impart or exacerbate “panty lines” visible through outer clothing; to provide for suitably snug fit that holds the absorbent gusset closely against the wearer's body and avoids gapping at the leg openings with shifting body movements; to prevent wicking and leakage of liquid from side edges of the gusset; and to avoid puncturing the barrier layer and edge sealing structure of the gusset with, e.g., stitches, which can provide leakage pathways.

Referring to FIG. 4A, these objectives are served by the configuration illustrated. Gusset 230 including absorbent assembly 238 is affixed to the outward-facing layer 234 of crotch portion 130 by a strip or tape of adhesive 236, which may be of the type that may be heat-pressed between an outward-facing surface of the gusset (in the example shown, outward-facing surface of barrier layer 233) and outward-facing layer 234. The adhesive 236 is laterally inset from the leg opening edge 140 of the pant by an adhesive inset distance AI. Preferably, adhesive 236 does not overlap, underly or overly the lower portion 235L of edge sealing strip 235 (when present; note optional alternative gusset structure shown in FIG. 3C); its laterally outermost edge is disposed either at the edge of, laterally inboard from, portion 235L. This laterally inset positioning of adhesive 236 may be imparted along the entirety of the portion of gusset 230 proximate the nearest leg opening edge 140. This positioning reduces material stack-up in with the edge sealing strip 235, and also, removes the adhesive 236 from proximity with the leg opening edge 140, thus removing any stiffening effect and/or elasticity-reducing effect that adhesive 236 may have on the structure where it is applied. In some examples adhesive inset distance AI may have a minimum value of 2 mm, more preferably 4 mm, even more preferably 7 mm and still more preferably 10 mm along the entirety of the gusset edge proximate the leg opening edge 140. Where a range within tolerances supported by particular manufacturing methods is desired, the inset distance AI along each leg edge may be, e.g., from 2 mm to 20 mm, more preferably from 4 mm to 17 mm, and even more preferably from 7 mm to 14 mm. Where desired based upon material choice, a strip of fabric finishing trim 237 may be adhered to outward-facing layer 234 by trim adhesive 237a, to provide a more finished-looking edge to the pant structure at the leg opening edge 140. Referring to FIG. 4B, in another example, adhesive 236 may extend across the gusset to a greater extent, but still be inset from the leg opening edges by distance AI as shown. No stitching is required.

FIG. 5 illustrates another example configured to serve the objectives identified above. A pattern of discrete “micro-dots” of adhesive 236d may be disposed between the gusset 230 and the outward-facing layer 234 in any desired position, including between the lower portion 235L of edge sealing strip 235, and outward-facing layer 234. Adhesive in the form of a pattern of discrete micro-dots may be applied to a substrate material layer by, e.g. SEWFREE ON DEMAND equipment available from Bemis Associates Inc., Shirley, Massachusetts, USA. Other alternatives are commercially available, including patterns of adhesive micro dots that are applied to transfer/release strips or sheets (paper or film) in a configuration enabling them to be transferred to fabric via iron or press. One such alternative is sold as PINBOND transfer adhesive, a product of New Textile Technologies GmbH, Balingen, Germany. A pattern of discrete micro-dots of adhesive may be an effective fastening mechanism for purposes herein in that fastening strength is provided through numerosity of discrete, closely-spaced fastening points at each of the micro-dots, while at the same time the micro-dots of adhesive do not have a substantial binding or stiffening effect on stretchable layer substrates to which they are adhered, because the substrates may stretch and flex between the microdots. The pattern also will typically have a substantially lower basis weight of adhesive, compared to a continuous strip or tape of adhesive. Thus, the adhesive micro-dots can be effective to adhere respective superadjacent/subjacent materials together, but also help substantially mitigate material stack up, and avoid binding, stiffening, and reducing elasticity of the substrates that they serve to adhere together. No stitching is required.

As a supplement to, complement to, or substitute for, adhesive micro-dots, the manufacturer may prefer to join materials where adhesive might otherwise be used, via thermal compression bonding or ultrasonic bonding, for example, in the form of a pattern of discrete bonds. Such bonds and a pattern thereof may be arranged and function in a manner similar to a pattern of adhesive micro-dots.

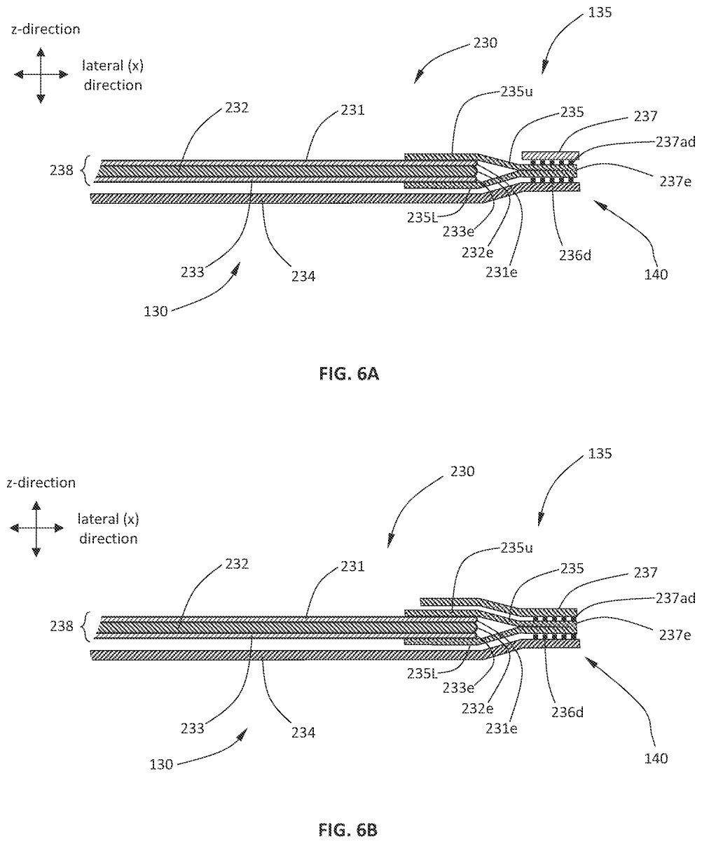

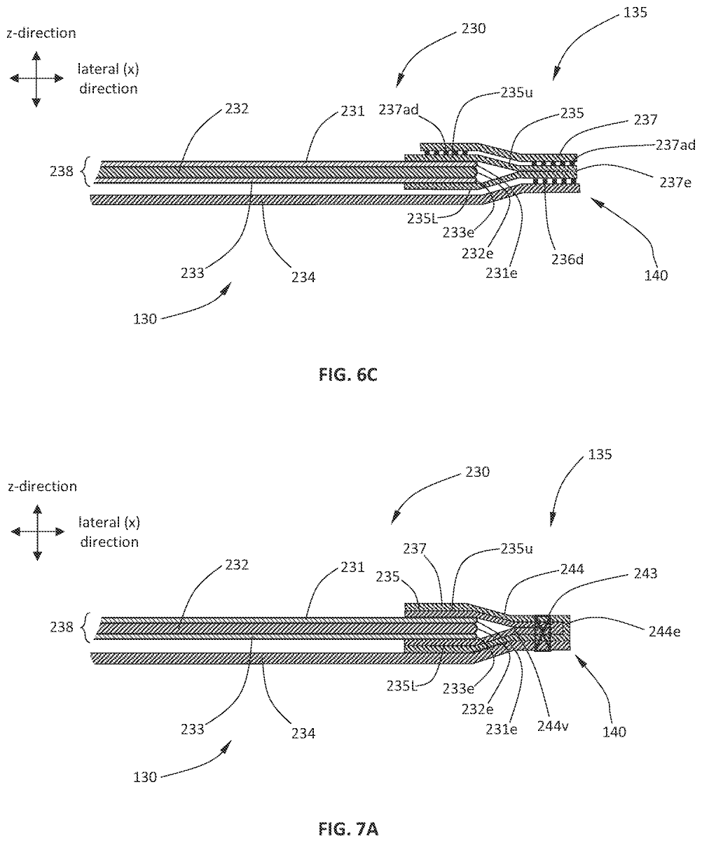

FIGS. 6A-6C illustrate additional examples configured to serve the objectives identified above. Edge sealing strip 235 is folded about itself in a “V” configuration about the peripheral edge of the absorbent assembly 238, and includes a sufficient margin of material that when pressed and adhered to itself outboard of the absorbent assembly 238 edges and proximate the fold, creates an extending portion 237e. Extending portion 237e may be attached to outward-facing layer 234 by micro-dot adhesive 236d as shown. As discussed above, the micro-dot adhesive 236d avoids material stack up, stiffening and loss of elasticity of the pant structure where it is used, in the examples shown, proximate the leg opening edges 140. These benefits may be exploited as they provide greater opportunity flexibility to include an elastically stretchable fabric trim strip 237 without substantial compromise to stretchability, elasticity and flexibility, to at least partially cover edge outer edges of sealing strip 235 and provide a finished appearance about the gusset edges. Trim strip 237 also may be adhered to the upper portion 235u of edge sealing strip by trim adhesive in micro-dot form, 237ad, as described above.

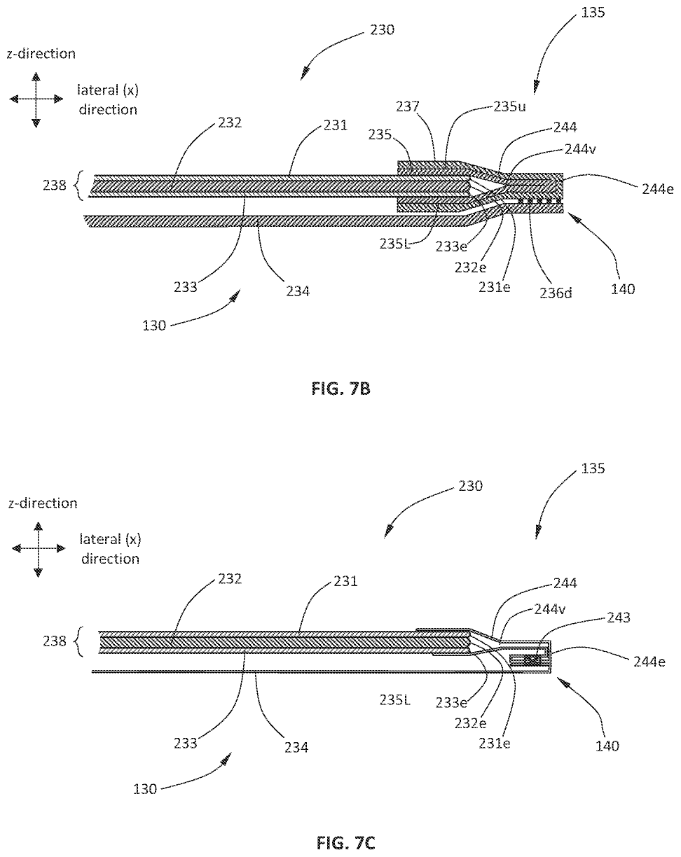

FIGS. 7A-7C illustrate additional examples configured to serve the objectives identified above. An elastic edge sealing material 235 and an extensible fabric trim material 237 may be combined as a laminate strip, which may then be folded over on itself along its length to form a combination “V”-shaped strip 244, with legs of the “V” respectively adhered to the wear-facing and outward-facing surfaces of the absorbent assembly 238. Sufficient margin may be provided outboard of the perimeter edges of assembly 238, to provide an extending flange 244e. Extending flange 244e may then be affixed to outward-facing layer 234 by any suitable mechanism, such as by stitching 243 (FIG. 7A) or micro-dot adhesive (FIG. 7B).

Where stitching is used to attach the gusset assembly 230 to the outward-facing layer 234 as suggested in FIG. 7A, the stitching is preferably of a stitch pattern that provides for extensibility along the path of the stitching. A variety of examples of potentially suitable patterns are known in the garment-manufacturing field, including, for example, zig zag stitch, lightning bolt stitch, triple straight stitch, twin needle stitch, serge stitch, blind hem stretch stitch, honeycomb stitch, feather stretch stitch, etc. It may be appreciated from FIG. 7A that, although the stitches penetrate the edge sealing material 235, the margin of extra material of the laminate 244 enables the edge sealing material 235 component thereof to be adhered to or be in sealing contact with itself inside the “V” structure, at a location 244v inboard of the stitching 243, thereby eliminating leakage pathways to the stitching 243.

In another example illustrated in FIG. 7B, the gusset 230 may be affixed to outward-facing layer 234 with micro-dot adhesive 236d, as described above.

FIG. 7C, simplified for purposes of visual clarity, illustrates another possible example, in which a seam 135 formed by stitches 243 joining gusset assembly 230 and outward-facing layer 234 together are concealed. This configuration may be formed by first stitching gusset 230 through extending flange formed of laminate 244, to outward-facing layer 234 along leg edges 14, while each are in an inverted or “inside out” configuration relative each other, while leaving the forward and rearward ends of the gusset unattached to the outward-facing layer. Next, the affixed gusset and outward-facing layer may be inverted, or turned “outside out,” to result in the configuration reflected in FIG. 7C, which internalizes and conceals the stitching 243 and presents a leg edge 140 having a hemmed and finished appearance. Although this configuration may appear to have an increased stack up of materials proximate the leg edges 140, selection of relatively low caliper, lower basis weight materials for the fabric trim material layer of laminate 244 can have a mitigating effect. Further, as can be seen, the materials forming the absorbent assembly 238 of the gusset do not underly or overlie the seam 135 joining the gusset 230 to the outward-facing layer 234 proximate the leg edges 140, and so do not contribute to material stack up at the leg opening edges. (This avoidance of stack up at the leg opening edges, by absorbent assembly 238, is generally true for all examples shown and described herein—in all examples, the perimeter edges of absorbent assembly 238, i.e., perimeter edges of layers 231, 232 and 233, are disposed laterally inboard of the leg opening edges 140.)

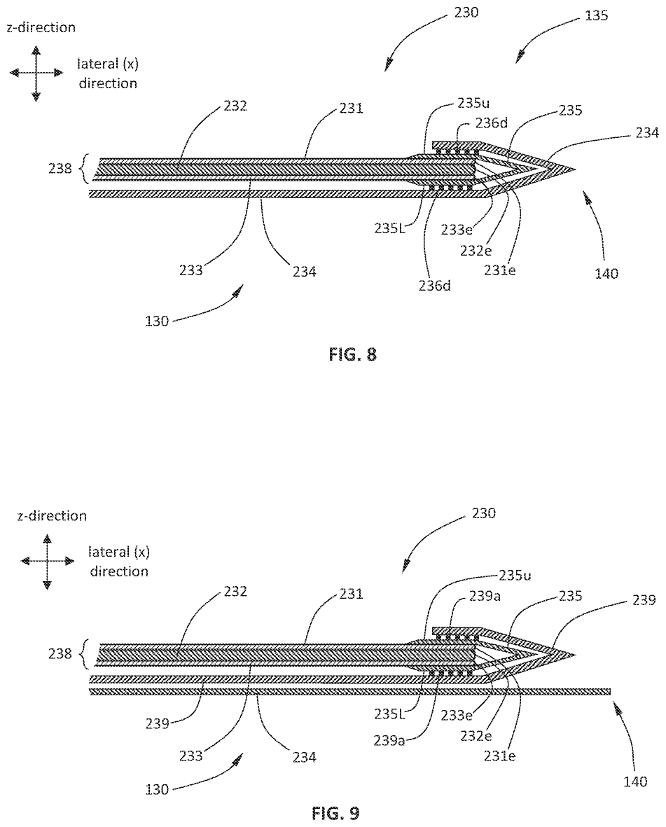

FIG. 8 reflects yet another approach. In the reflected configuration, an extra margin of outward-facing layer 234 is provided to wrap up and fold around the folded perimeter edge of edge sealing strip 235, to provide a leg opening edge 140 having a finished appearance. Deposits of micro-dot adhesive 236d may be used to adhere the gusset 230 to the outward-facing layer 234 one or more of the locations shown and/or other locations, but at least at the upper location shown, proximate the wrapped edge of outward-facing layer 234.

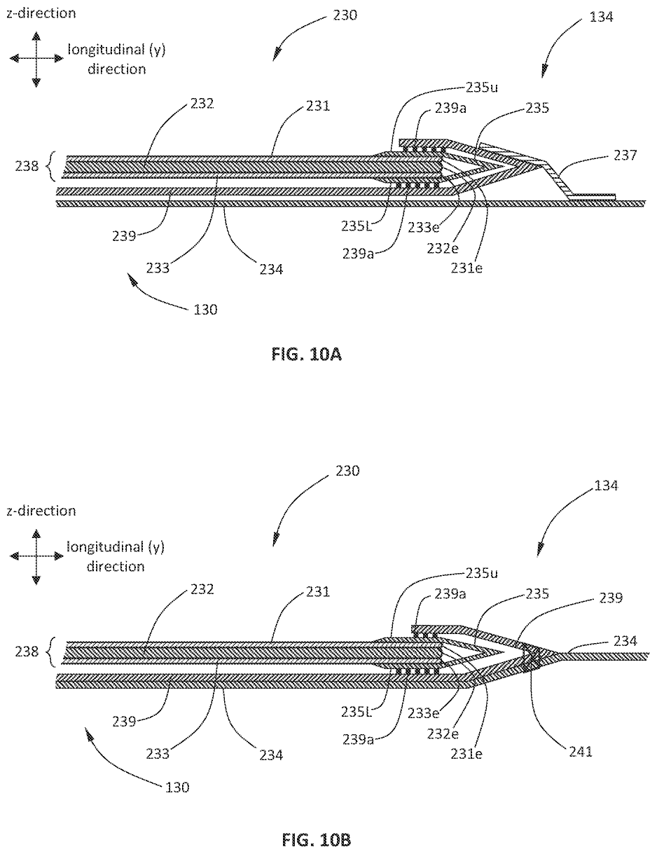

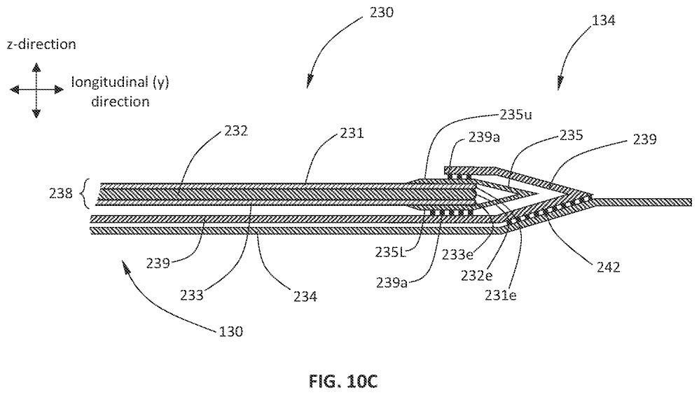

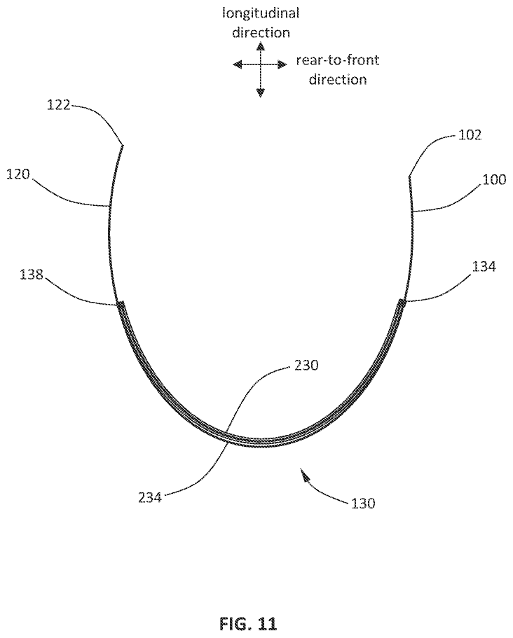

FIG. 9, viewed in conjunction with FIGS. 10A-10C and 11, reflects another approach, referred to herein as a “hammock” configuration. In this configuration, the gusset assembly 230 may be unaffixed to the outward-facing layer 234 of the pant in the crotch portion 130, and/or unaffixed to the outward-facing layer 234 along or proximate the leg edges 140, or even affixed only at forward and rearward ends, along seams 134 and 138 (shown in FIG. 2). Between the seams 134 and 138, the gusset may be unaffixed to the outward-facing layer 234. This de-coupling of the gusset 230 and outward-facing layer 234 allows the outward-facing layer 234 and any elastomeric/elastic components thereof, or affixed thereto, to move and flex independently of the components of the gusset 230, between the forward and rearward seams 134, 138, and provide better ability of the outer pant structure including outward-facing layer 234 and any associated leg band components to urge the gusset assembly against the wearer's body, particularly in configurations in which the gusset assembly might not be as stretchable/elastically extensible as the outer pant structure including outward-facing layer 234. Further, the hammock configuration enables the wearer, if she desires to wear the underpant with a supplemental sanitary napkin of the type having side “wings”, to wrap the wings over the side/leg edges of the gusset and then underneath it, between the gusset and the outward-facing layer 234, thereby concealing the wings behind the outward-facing layer. Depending upon whether the gusset assembly is more or less elastically extensible in a longitudinal direction as the underlying outer pant structure/outward-facing layer 234, it may be desired that the relaxed/contracted length of the gusset assembly between seams 134, 138 be equal to or greater than, or less than, the corresponding relaxed/contracted length of the underlying outward-facing layer(s) 234 between the seam 134, 138 locations. This may be desired to help ensure that the outer pant structure including outward-facing layer 234 is always in longitudinal tension, to help urge the gusset assembly against the wearer's body, through shifting body movements. With respect to FIG. 11, the respective lengths will be the lengths of outward-facing layer 234 and gusset assembly 230, between longitudinally innermost extents of seams 134 and 138 at the longitudinal axis 200 of the pant.

As reflected in FIG. 9, the hammock configuration may include added gusset backing layer 239, disposed beneath the barrier layer. Gusset backing layer 239 may be formed of the same fabric as outward-facing layer 234, or of a differing fabric material. Gusset backing layer 239 may be included to cover, hide and protect barrier layer 233, which in some examples may be fragile and/or may have an undesirably tacky or plastic-like feel and appearance, which the manufacturer may wish to conceal from the wearer's view and touch. In some examples, gusset backing layer 239 may be formed of a fabric that is effectively waterproof (highly resistant to passage of fluid therethrough, under conditions of use contemplated herein—like barrier layer 233 as described above). In such examples, gusset backing layer 239 may supplement or even substitute for and supplant barrier layer 233. Gusset backing layer 239 may be affixed to the absorbent assembly 238 and/or edge sealing strip 235 at the locations shown, by hammock adhesive 239a. Proximate leg edges 140, it may be desired that hammock adhesive 239a be micro-dot adhesive for the reasons explained above, while hammock adhesive 239a may be micro-dot adhesive, or tape or strip adhesive, along forward and rearward gusset seams 134, 138.

FIGS. 10A-10C schematically illustrate longitudinal cross sections of possible examples of configurations for forward and/or rearward seams 134, 138 affixing the gusset assembly 230 to the outward-facing layer 234 in the front 100 and rear 120 portions of the pant.

As reflected in FIG. 10A, a fabric trim strip 237 may be disposed to have one side overlay a forward or rearward end of the gusset 230, and a second side to overlay the outward-facing layer 234 of the pant structure. Trim strip 237 may be affixed at these locations via any suitable mechanism such as adhesive, micro-dot adhesive, stitching, etc. It will be appreciated that concerns for material stacking, binding, stiffening and loss of elasticity may not be as great for forward and rearward seams 134, 138, as for the regions of the pant proximate the leg openings, including leg opening edges 140 in the crotch portion 130.

FIGS. 10B and 10C reflect a more simplified construction, in which material of the gusset backing layer 239 is directly affixed to outward-facing layer 234, at a location that is longitudinally outboard of the edge sealing strip 123 but longitudinally inboard of the folded-over edge of the hammock layer. As reflected in FIG. 10B, the two layers of folded-over gusset backing layer 239 may be stitched with gusset end stitching 241 to the outward-facing layer 234, to form seam 134 (or for a rearward seam, 138). As reflected in FIG. 10c, the outward-facing portion of gusset backing layer 239 may be adhered to the outward-facing layer 234 along seam 134 (or for a rearward seam, 138) by a path of micro-dot gusset end adhesive 242. Noting, however, that concerns for material stacking, binding, stiffening and loss of elasticity may not be as great for forward and rearward seams 134, 138, a tape or strip adhesive may be substituted for micro-dot adhesive 242.

With respect to any of the examples depicted in FIGS. 4A through 6C, 7B, 8, 9, 10A and 10C, it will be noted that the designs reflected do not include stitched seams, but rather, rely upon adhesive, micro-dot adhesive or ultra-sonic bonding to join layers. In some examples of such designs, areas at or proximate the corners of the gusset may be vulnerable to multi-directional stresses that may precipitate separation of the material layers that are adhered or bonded, at the corners. Accordingly, referring back to FIG. 2, it may be desired to include tack stitches 245 proximate to two or all four of the gusset corners, that penetrate and join at least the outward-facing layer 234 and the gusset structure, directly, or indirectly, e.g., via/through a layer overlying wearer-facing layer 231. Such tack stitches, being discrete and localized proximate the gusset corners, will not substantially impair elastic stretch of the materials they join. Where such tack stitches are included, to avoid providing potential leakage pathways from layers to/through which fluids may wick, it may be preferred that the tack stitches do not contact and/or do not penetrate or extend through one or more of absorbent layer(s) 232 and wearer-facing layer 231. For purposes of minimizing their potential impact upon stretch or potential for causing puckering of outward-facing layer 234, such tack stitches, where included, preferably have a largest x-y plane dimension no greater than 15 mm, more preferably no greater than 12 mm, and even more preferably no greater than 10 mm, and still more preferably no greater than 8 mm.

Other Construction Details

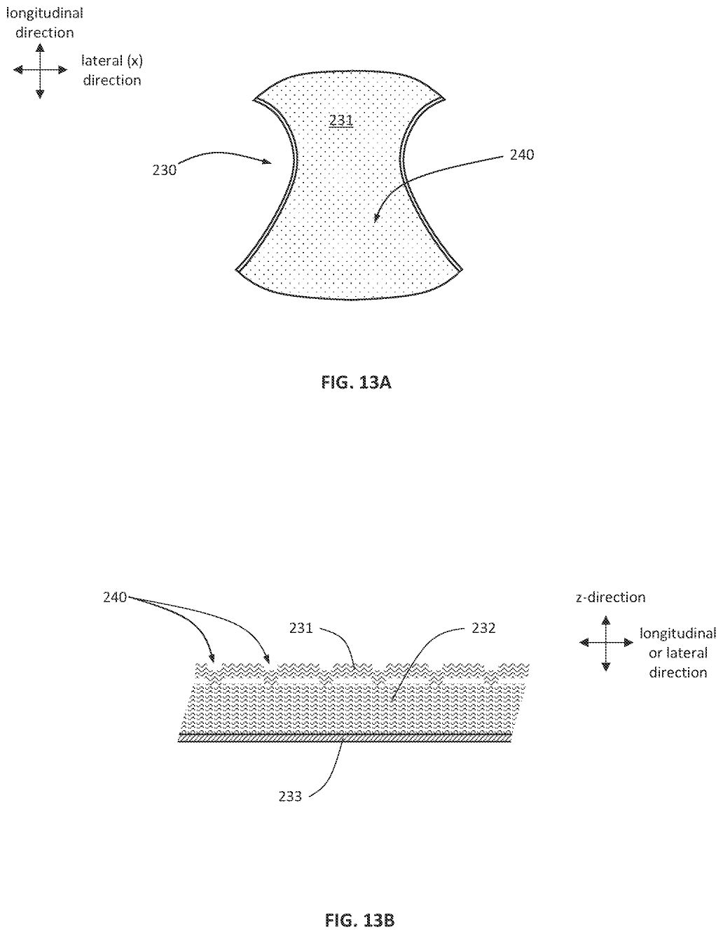

In some examples one or more layers within the crotch portion 130 and/or gusset 230 may be bonded together via any suitable bonding mechanism. Referring to FIGS. 13A and 13B for example, a wearer-facing layer 231 may be bonded to a subjacent absorbent layer 232 for purposes of holding the layers in close proximity and thereby helping maintain a desired low caliper of the structure, and enhancing fluid communication between the layers, i.e., enhancing the structure's ability to pass discharged urine or menstrual fluid from the wearer-facing layer to the underlying absorbent layer, via contact between the layers. In such example, the bonding mechanism should be selected so as not to occlude the interface between the two layers along any substantial portion of their interfacing surface areas. Thus, in some examples it may be desired that the bonding mechanism have the form of a discontinuous pattern of discrete bonds (i.e., a pattern with unbonded regions between the bonds), such as a regular pattern of spot bonds 240. In some examples, bonds between the layers may be formed by discrete deposits of adhesive between the layers, adhering them together at the locations of the deposits. In other examples, bonds between the layers may be formed by thermal compression bonding. The latter bonding mechanism may be more durable through a plurality of launderings, and as illustrated schematically in FIG. 14B, creates a corresponding pattern of z-direction depressions in the wearer-facing layer 231 that may serve to collect discharged urine/fluid and initiate and/or facilitate its movement (via wicking) down to the absorbent layer 232. For purposes of enabling thermal compression bonding, the wearer-facing layer and the subjacent absorbent layer may each include polymer components of respective fusible compositions (such as similar polyester-based compositions) that facilitate formation of robust bonds between the layers upon localized application of heat and pressure at the bond sites. (Herein, “fusible compositions,” with respect to two respective polymers present in two respective layers, means that the two polymers are miscible, capable of melting and mixing at a temperature of 250° C. or lower, to form a single thermodynamic phase.)

Various layers that may be included in crotch portion 130 and/or crotch gusset 230, e.g., layers 231, 232, 233 and 234, may also be joined to each other by any suitable mechanism at forward and rearward seams 134, 138 and crotch side seams 135 proximate the leg edges 140. The joining mechanism may be a system of stitching to affix the layers together; however, for purposes of liquid containment it may be desired that the joining mechanism include a generally hydrophobic, water insoluble adhesive or adhesive film, by itself or as a supplement to stitching, or alternatively, that such stitching be entirely above the barrier layer (not penetrating the barrier layer) or within the containment zone of the edge sealing strip 235.

Measurement Methods

General Sample Preparation

Unless otherwise specified below, each of the measurements below is to be conducted on 10 separate like samples (taken from 10 separate like examples of pants) and the average of the 10 separate like samples is considered to be the measurement for that specific sample set.

Referring to FIG. 2, samples including the entire crotch gusset 230 are collected from examples of the subject pant. Lateral lines 234, 238 that are respectively tangent the forwardmost edge of the forward seam 134 and rearward-most edge of the rearward seam 138 are identified, and the pant is cut apart along these lines (without cutting into the seam itself) to provide a sample that includes the entire crotch gusset 230.

If the pant is a legged pant, cut out the gusset in its entirety, along cutting paths outside of the seam(s) joining the gusset to the remainder of the pant, without cutting into the seams themselves. For legged pants, measurements of “Crotch Width” made for purposes herein will be the width of the removed gusset measured along the lateral direction, along a lateral line marking the shortest distance between the leg openings prior to removal of the gusset from the legged pant.

The sample should be cut from the example pant with a sharp knife or suitably sharp cutting device effective to precisely and cleanly cut the sample. A straight edge or other suitable drafting/drawing tool may be used where helpful to hold the example down on the work surface and help guide the cutting device.

The testing is performed under ambient room conditions (temperatures from between 15° C. to 35° C. and relative humidity from between 35% to 75%). Samples are conditioned for at least two hours prior to testing under the same conditions.

All linear dimensions are measured manually by ruler within the ordinary x-y plane, using a ruler that is traceable to NIST or other standards organization.

Longitudinal Tensile Modulus

For the Longitudinal Tensile Modulus and Maximum Longitudinal Elongation test methods, the samples as described above in “General Sample Preparation” are further modified by cutting out a 40 mm laterally wide section symmetrically about the longitudinal axis 200. The leg edge seams are not included in these tests. If the lateral width between the narrowest separation of leg edge seams as defined by the tangent lines 270 in FIG. 2 is less than 40 mm, then the pant is not deemed to be suitable (as being too narrow through the crotch region for suitable coverage) for purposes described herein, and is considered to be outside the scope of the claims.

The Longitudinal Tensile Modulus of the sample is determined by stretching along the direction of the longitudinal axis 200 of the pant, using a constant rate of extension tensile testing machine with computer interface, e.g., Instron; MTS; Zwick; etc., using a load cell for which the loads measured are within 10% to 90% of the limit of the cell, and ensures accuracy of a 5N load to 0.1N. The instrument is equipped with a single line contact grips, 8 cm in grip width. Prior to testing, calibrate the equipment according to the instruments manufacturer's recommendations.

In accordance with the sample preparation instructions set forth above, the Sample Width is 40 mm. The stress in the sample is calculated by dividing the force in the load cell by the Sample Width, and is expressed in units of gf (grams-force)/mm. (The caliper of the sample is not a factor in this calculation.) The Sample Length is equal to the length of the sample along the longitudinal axis 200 between the lateral lines 234, 238 (along which the sample was cut from the example pant), as illustrated by way of example in FIG. 2.

The grips of the tensile testing machine consist of air actuated grips designed to hold the sample. No slippage should be permitted between the sample and the grips. The distance between the grips (along the axis of the machine's elongation) should be the Sample Length minus 6 cm. This distance will be hereinafter referred to as the “Starting Gauge-Length”.

The sample is mounted in the grips with its longitudinal axis 200 parallel to the direction of applied elongation, and centered in each grip. Two (2) cm of the sample's length at each end is inserted into each grip. The Starting Gauge Length, determined as described above, will ensure that 2 cm of longitudinal slack will be present in the sample at the start of the test.

After the sample is mounted, the machine's load channel is set to zero (this eliminates the weight of the sample in the calculations). The grips are slowly moved apart at 5.08 cm/min (2.0 in/min) until a load of 5 gf (grams-force) is reached. The separation between the grips at this position is recorded as L0.

(L0=Starting Gauge Length+additional machine extension to reach 5 gf)

After the 5 gf load is reached, extend the sample at a rate of 50.8 cm/min (20 in/min) with a data acquisition rate of 50 Hz. Extend until either a stress of 30 gf/mm is reached, or the sample breaks.

Sample strain is calculated by ΔL/L0. ΔL is any additional extension between the grips after L0 is reached and is recorded along with load at a rate of 50 Hz. Sample strain is expressed numerically (not as a percentage), thus a strain of 100% is 1.0 for the purposes of these calculations.

Record the sample strains at sample stresses of 10 gf/mm and at 20 gf/mm.

Longitudinal Tensile Modulus is the linear slope between 10 gf/mm and 20 gf/mm, and is calculated as:

Longitudinal Tensile Modulus=[20 gf/mm−10 gf/mm]/[sample strain at 20 gf/mm−sample strain at 10 gf/mm]
Repeat for 10 samples.

Maximum Longitudinal Elongation

Maximum Longitudinal Elongation is measured during the Longitudinal Tensile Modulus test. The Maximum Longitudinal Elongation is the sample strain at a sample stress of 20 gf/mm. Maximum Longitudinal Elongation is expressed as a percent strain, e.g., a value of 1.0 strain from the Longitudinal Tensile Modulus method is expressed as 100% strain for Elongation.

Repeat and record the results for 10 samples. Calculate and record the average of the results. The average will be the Maximum Longitudinal Elongation value for the subject pant design.

Absorption Capacity

The absorption capacity test measures the amount of liquid held within a test sample after specified times of immersion and vertical drainage. The amount of test liquid that is retained by the test sample is used to calculate and report the Area Absorption Capacity (milliliters (ml) of liquid per specimen area in square centimeters) and the Volume Absorption Capacity (in milliliters (ml) of liquid per specimen volume in cubic centimeters). All testing is performed in a room controlled at 23° C.±3° C. and 50%±2% relative humidity.

The test procedure follows compendial method WSP 010.1.R3 (12) part B (Liquid Absorptive Capacity) with modifications specified as follows. The test liquid is deionized water at room temperature (23° C.±3° C.; density 1.00 g/ml). For the weighing portions of the test, no cover glass is used as the test liquid is non-volatile. The overall dimensions of the wire gauze test specimen support is large enough (e.g. 12 inches by 12 inches) to accommodate the larger test sample size. The test sample is the entire gusset (as described herein), thus larger than what is suggested in the compendial method.

Prior to measuring absorption capacity, the examples of the pants of interest (prior to removal of samples therefrom) are washed in order to mimic in-use conditions and to follow the recommended “prior to use” instructions that accompany these types of pants (e.g. wash before use). The examples are placed into a mesh lingerie bag, and then placed into a high efficiency, front-loading washing machine (any convenient source) along with a single small/light load dosage of TIDE brand laundry detergent (“Original” designation; “HE” or other high efficiency washing machine designation; without additives such as FEBREZE, ODOR DEFENSE, OXI additives, bleach or bleaching additives or fabric softening additives) (product of The Procter & Gamble Company, Cincinnati, Ohio). The washer is set to delicate cycle using cold water. After the wash cycle, the examples are removed from the mesh bag and placed flat on a drying rack to air dry for about 12 hours. After air-drying, the examples are placed into a clothes dryer (any convenient source) set on the delicate cycle with very low heat for about 5 minutes or until dry to the touch. Test samples are prepared as follows. The pre-washed and dried examples are equilibrated in a room controlled at 23° C.±3° C. and 50%±2% relative humidity for about 2 hours. Test samples that include the entire gusset are removed from the examples as described in the General Sample Preparation section herein. Using scissors, the outermost fabric layer on the outward-facing side of the gusset is cut out along the entire gusset shape, inboard of the seams, using care so as not to cut into any of the edge seams present.

The immersion and drainage procedure outlined in the compendial method is then followed with the modifications previously noted. Subtract the Dry Mass from the Wet Mass and record as Liquid Mass Absorbed to the nearest 0.01 grams. Since the density of deionized water is 1.00 g/ml, the Liquid Mass Absorbed is also recorded as Liquid Volume Absorbed to the nearest 0.01 ml. Divide the Liquid Volume Absorbed (ml) by the overall area (cm²) of the test specimen and record as Area Absorption Capacity to the nearest 0.01 ml/cm². Now divide the Liquid Volume Absorbed (ml) by the volume of the test specimen (area×central caliper) and record as Volume Absorption Capacity to the nearest 0.01 ml/cm³.

In like fashion, repeat for a total of three replicate test specimens. Calculate the arithmetic mean for Area Absorption Capacity and Volume Absorption Capacity and report to the nearest 0.01 ml/cm² and 0.01 ml/cm³, respectively.

Caliper

The Caliper of a sample including a crotch gusset is measured as the distance between a reference platform on which the sample rests and a pressure foot that exerts a specified amount of pressure onto the sample over a specified amount of time. All measurements are performed in a laboratory maintained at 23° C.±2° C. and 50%±2% relative humidity.

Caliper is measured with a manually-operated micrometer equipped with a pressure foot capable of exerting a steady pressure of 0.5 kPa±0.01 kPa onto the test sample. The manually-operated micrometer is a dead-weight type instrument with readings accurate to 0.001 mm. A suitable instrument is Mitutoyo Series 543 ID-C Digimatic, available from VWR International, or equivalent. The pressure foot is a flat ground circular movable face with a diameter of 50 mm. The sample is supported by a horizontal flat reference platform that is larger than and parallel to the surface of the pressure foot. The system is calibrated and operated per the manufacturer's instructions.

Obtain a sample including the gusset by removing it from the pant, as described above. When excising the sample from an absorbent article, use care to not impart any wrinkles into the layers or other distortion of the layers during the removal process. Samples are conditioned at 23° C.±2 C.° and 50%±2% relative humidity for 2 hours prior to testing. To measure caliper, first zero the micrometer against the horizontal flat reference platform. Place the sample on the platform with the desired measurement location centered below the pressure foot. Gently lower the pressure foot with a descent rate of 1.0 mm±0.1 mm per second until the full pressure is exerted on the sample. Wait 5 seconds and then record the caliper of the sample to the nearest 0.01 mm. In like fashion, repeat for a total of 10 replicate samples. Calculate the arithmetic mean for the Caliper and report to the nearest 0.01 mm.

Liquid Impermeability

When there is a question concerning whether a particular pant has a gusset with a liquid impermeable barrier layer, this Liquid Impermeability test method may be used to measure a quantity of test liquid that will pass through a sample and enable determination whether there is a “liquid impermeable barrier layer” present in the gusset, according to the definition set forth in the description above.

The Liquid Impermeability test measures the quantity of liquid transferred through to the outward-facing side of a test specimen obtained from a pant after it is dosed with a prescribed volume of test liquid in order to simulate a liquid insult during actual use/wear of the pant.

All testing is performed in a room controlled at 23° C.±3° C. and 50%±2% relative humidity.

Prior to performing the measurement of this method, the examples of the pants of interest (prior to removal of samples therefrom) are washed in order to mimic in-use conditions and to follow the recommended “prior to use” instructions that accompany these types of pants (e.g. wash before use). The examples are placed into a mesh lingerie bag, and then placed into a high efficiency, front-loading washing machine (any convenient source) along with a single small/light load dosage of TIDE brand laundry detergent (“Original” designation; “HE” or other high efficiency washing machine designation; without additives such as FEBREZE, ODOR DEFENSE, OXI additives, bleach or bleaching additives or fabric softening additives) (product of The Procter & Gamble Company, Cincinnati, Ohio), or equivalent. The washer is set to delicate cycle using cold water. After the wash cycle, the examples are removed from the mesh bag and placed flat on a drying rack to air dry for about 12 hours. After air-drying, the examples are placed into a clothes dryer (any convenient source) set on the delicate cycle with very low heat for about 5 minutes or until dry to the touch.

Test samples are prepared as follows. The pre-washed and dried example pants are equilibrated in a room controlled at 23° C.±3° C. and 50%±2% relative humidity for about 2 hours. Test samples containing the entire gusset are removed from the examples as described in the General Sample Preparation section herein. Using scissors, the outermost fabric layer on the outward-facing side of the gusset is cut out along the entire gusset shape, inboard of the seams, using care so as not to cut into any of the edge seams present. Mark the dose location at the intersection of the midpoint of the longitudinal axis of the sample and a lateral axis positioned at the narrowest portion of the specimen.

For each test sample, a single layer of filter paper is cut to 10 cm by 2.54 cm. A suitable filter paper is Ahlstrom Grade 989 (available from Ahlstrom-Munksjo North America LLC, Alpharetta, GA), or equivalent. The test liquid is deionized water at room temperature (23° C.±3° C.).

Record the mass of one layer of pre-cut filter paper and record as Dry Massfp to the nearest 0.0001 grams. Place the pre-weighed filter paper onto a flat horizontal work surface. Position the test specimen centered over the filter paper with the garment facing side of the specimen facing the paper. Using a volumetric pipette, apply a 1.0 ml dose of test liquid to the pre-marked dosing location as follows. The tip of the pipette is held about 3 mm above the surface of the test specimen, and the dose is applied slowly (about 30 seconds) to avoid splashing. As soon as the entire dose has been applied, start a 1 minute timer. After 1 minute has elapsed, remove the test specimen and record the mass of the filter paper as Wet Massfp to the nearest 0.0001 grams. Subtract the Dry Massfp from the Wet Massfp and record as z-Direction Leakage to the nearest 0.0001 grams.

In like fashion, repeat for a total of three replicate test specimens. Calculate the arithmetic mean for z-Direction Leakage and report to the nearest 0.0001 g.

Ordinary X-Y Plane Dimensions

For purposes herein, when a length or width of a feature of a pant is specified, it is to be measured with the pant laid out flat on a horizontal planar surface (in an opened or assembled configuration, as appropriate) with the material of the pant smoothed out flat, but in a relaxed condition, not pulled or stretched along any planar direction.

In view of the foregoing description, the following examples are contemplated, although the identification thereof is not intended to be limiting of or on any claims to be asserted, to any combinations of any features identified and/or described herein.

1. A durable absorbent pant, comprising:
an outer pant structure comprising:
   a front waist portion (100) with a front waist edge (102);
   a rear waist portion (120) with a rear waist edge (122);
   a crotch portion (130) having a forward portion (132) meeting the front waist portion and a rearward portion (136) meeting the rear waist portion; and
   left and right hip side portions (160) at which the front waist portion meets the rear waist portion to form a waist opening with a waist opening edge comprising the front waist edge and the rear waist edge, and to form left and right leg openings with left and right leg opening edges (104, 140, 124); and
   a gusset (230) disposed at least partially in the crotch portion, the gusset having a wearer-facing surface, an outward-facing surface and a perimeter edge, and comprising:
   an absorbent layer (232);
   a liquid-impermeable barrier layer (233) disposed beneath the absorbent layer, the barrier layer comprising an outward-facing surface of the gusset assembly; and
   an edge barrier that overlies portions of the wearer-facing surface proximate the perimeter edge within the crotch portion and effectively continues over and around the perimeter edge from the wearer-facing surface to the outward-facing surface in the crotch region, wherein the edge barrier comprises an extension of the barrier layer (233);
   wherein the outward-facing surface of the absorbent gusset assembly is affixed within the crotch portion (130) by one or more deposits of adhesive (236) disposed laterally inboard of the respective proximate left and right leg opening edges (140) by an adhesive inset distance (AI) of at least 2 mm, more preferably 4 mm, even more preferably 7 mm and still more preferably 10 mm.

2. A durable absorbent pant, comprising:
an outer pant structure comprising:
   a front waist portion (100) with a front waist edge (102);
   a rear waist portion (120) with a rear waist edge (122);
   a crotch portion (130) having a forward portion (132) meeting the front waist portion and a rearward portion (136) meeting the rear waist portion; and
   left and right hip side portions (160) at which the front waist portion meets the rear waist portion to form a waist opening with a waist opening edge comprising the front waist edge and the rear waist edge, and to form left and right leg openings with left and right leg opening edges (104, 140, 124); and
   a gusset (230) disposed at least partially in the crotch portion, the gusset having a wearer-facing surface, an outward-facing surface and a perimeter edge, and comprising:
   an absorbent layer (232);
   a liquid-impermeable barrier layer (233) disposed beneath the absorbent layer, the barrier layer comprising an outward-facing surface of the gusset assembly; and
   an edge barrier that overlies portions of the wearer-facing surface proximate the perimeter edge within the crotch portion and effectively continues over and around the perimeter edge from the wearer-facing surface to the outward-facing surface in the crotch region, wherein the edge barrier comprises one or more section(s) of liquid impermeable edge sealing strip material (235) joined to the barrier layer proximate the perimeter edge;
   wherein the outward-facing surface of the absorbent gusset assembly is affixed within the crotch portion (130) by one or more deposits of adhesive (236) disposed laterally inboard of the edge sealing strip material in the crotch portion.

3. The durable absorbent pant of either of the preceding examples wherein there is no deposit of adhesive affixing the absorbent gusset assembly within the crotch portion within less than 1.0 cm of a proximate left or right leg opening edge, that is continuous along a majority of the portion of the proximate leg opening edge within the crotch portion.

4. A durable absorbent pant, comprising:
an outer pant structure comprising:
   a front waist portion (100) with a front waist edge (102);
   a rear waist portion (120) with a rear waist edge (122);
   a crotch portion (130) having a forward portion (132) meeting the front waist portion and a rearward portion (136) meeting the rear waist portion; and
   left and right hip side portions (160) at which the front waist portion meets the rear waist portion to form a waist opening with a waist opening edge comprising the front waist edge and the rear waist edge, and to form left and right leg openings with left and right leg opening edges (104, 140, 124); and
   a gusset (230) disposed at least partially in the crotch portion, the gusset having a wearer-facing surface, an outward-facing surface and a perimeter edge, and comprising:
   an absorbent layer (232);
   a liquid-impermeable barrier layer (233) disposed beneath the absorbent layer, the barrier layer comprising an outward-facing surface of the gusset assembly; and
   an edge barrier that overlies portions of the wearer-facing surface proximate the perimeter edge within the crotch portion and effectively continues over and around the perimeter edge from the wearer-facing surface to the outward-facing surface in the crotch region, wherein the edge barrier comprises one or more section(s) of liquid impermeable edge sealing strip material (235) joined to the barrier layer proximate the perimeter edge, or, wherein the edge barrier comprises an extension of the barrier layer (233);
   wherein the gusset assembly (230) is affixed within the crotch portion (130) by a pattern of discrete deposits of adhesive (236d).

5. The absorbent example 4 wherein the deposits of adhesive (236d) in the pattern have an average size of 0.25 mm 2 to 2.0 mm².

6. The pant of either of examples 4 or 5, wherein an area of an outward-facing surface of the gusset assembly, entirely surrounded by but longitudinally and laterally inboard of the perimeter edge, is not affixed to the crotch portion.

7. The absorbent pant of any examples 4-6 wherein the deposits of adhesive (236d) in the pattern have the form of substantially circular dots.

8. The absorbent pant of any examples 5-7 wherein the pattern of deposits of adhesive (236d) follow paths that generally parallel the paths of the leg opening edges and are disposed inboard thereof and proximate thereto.

9. The absorbent pant of examples 4-8 wherein the pattern of deposits of adhesive (236d) reflects programmed contours conforming to the paths of the leg opening edges.

10. The absorbent pant of any of example 4-9 wherein the deposits of adhesive (236d) affix one or both the barrier layer (233) and edge barrier structure directly to the crotch portion (130).

11. A durable absorbent pant, comprising:
an outer pant structure comprising:
   a front waist portion (100) with a front waist edge (102);
   a rear waist portion (120) with a rear waist edge (122);
   a crotch portion (130) having a forward portion (132) meeting the front waist portion and a rearward portion (136) meeting the rear waist portion; and
   left and right hip side portions (160) at which the front waist portion meets the rear waist portion to form a waist opening with a waist opening edge comprising the front waist edge and the rear waist edge, and to form left and right leg openings with left and right leg opening edges (104, 140, 124); and
   a gusset (230) disposed at least partially in the crotch portion, the gusset having a wearer-facing surface, an outward-facing surface and a perimeter edge, and comprising:
   an absorbent layer (232);
   a liquid-impermeable barrier layer (233) disposed beneath the absorbent layer, the barrier layer comprising an outward-facing surface of the gusset assembly; and
   an edge barrier that overlies portions of the wearer-facing surface proximate the perimeter edge within the crotch portion and effectively continues over and around the perimeter edge from the wearer-facing surface to the outward-facing surface in the crotch region, wherein the edge barrier comprises one or more section(s) of liquid impermeable edge sealing strip material (235) joined to the barrier layer proximate the perimeter edge, or, wherein the edge barrier comprises an extension of the barrier layer (233);
   an extending flange 244e comprising a fabric material having a width, the entirety of the width being bonded to the edge barrier structure, the flange extending laterally outboard beyond a proximate side edge of the absorbent layer in the crotch portion, the extending flange being affixed to the outer pant structure.

12. The absorbent pant of any of the preceding examples wherein the gusset (230) comprises an elastic stretch knit fabric.

13. The absorbent pant of any of the preceding examples wherein the barrier layer (233) comprises an elastomeric film.

14. The absorbent pant of any of the preceding examples wherein the edge barrier comprises an elastomeric film.

15. The absorbent pant of any of the preceding examples comprising a supplemental elastic stretch knit fabric trip strip (237) having a first strip portion affixed to the gusset at a first attachment location and a second strip portion affixed to the front or rear waist portion at a second attachment location, wherein the deposits of adhesive are present at either or both the first attachment location and second attachment location.

16. A durable absorbent pant, comprising:
an outer pant structure comprising:
   a front waist portion (100) with a front waist edge (102);
   a rear waist portion (120) with a rear waist edge (122);
   a crotch portion (130) having a forward portion (132) meeting the front waist portion and a rearward portion (136) meeting the rear waist portion; and
   left and right hip side portions (160) at which the front waist portion meets the rear waist portion to form a waist opening with a waist opening edge comprising the front waist edge and the rear waist edge, and to form left and right leg openings with left and right leg opening edges (104, 140, 124); and
   a gusset (230) disposed at least partially in the crotch portion, the gusset having a wearer-facing surface, an outward-facing surface and a perimeter edge having forward and rearward edge portions respectively in the front waist portion and rear waist portions, and left and right crotch edge portions, and comprising:
   an absorbent layer (232);
   a liquid-impermeable barrier layer (233) disposed beneath the absorbent layer, the barrier layer comprising an outward-facing surface of the gusset assembly; and
   an edge barrier that overlies portions of the wearer-facing surface proximate the perimeter edge within the crotch portion and effectively continues over and around the perimeter edge from the wearer-facing surface to the outward-facing surface in the crotch region, wherein the edge barrier comprises one or more section(s) of liquid impermeable edge sealing strip material (235) joined to the barrier layer proximate the perimeter edge, or, wherein the edge barrier comprises an extension of the barrier layer (233);
   wherein the gusset (230) is affixed to the outer pant structure in the front and rear waist portions, respectively, not affixed to the outer pant structure in the crotch portion.

17. The absorbent pant of example 16 wherein:
the gusset (230) is affixed to the outer pant structure at forward and rearward seams (134, 138)
the gusset has a first length between the seams, when in a relaxed condition;
the outer pant structure has a second length between the seams, when in a relaxed condition; and
the first length is equal to or greater than the second length.

18. The absorbent pant of example 16 wherein:
the gusset (230) is affixed to the outer pant structure at forward and rearward seams (134, 138)
the gusset has a first length between the seams, when in a relaxed condition;
the outer pant structure has a second length between the seams, when in a relaxed condition; and
the first length is less than the second length.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed:

1. A durable absorbent pant, comprising:
an outer pant structure comprising:
a front waist portion with a front waist edge;
a rear waist portion with a rear waist edge;
a crotch portion having a forward portion meeting the front waist portion and a rearward portion meeting the rear waist portion; and
left and right hip side portions at which the front waist portion meets the rear waist portion to form a waist opening with a waist opening edge comprising the front waist edge and the rear waist edge, and to form left and right leg openings with left and right leg opening edges; and
a gusset assembly disposed at least partially in the crotch portion, the gusset assembly having a wearer-facing surface, an outward-facing surface and a perimeter edge, and comprising:
an absorbent layer;
a liquid-impermeable barrier layer disposed beneath the absorbent layer, the barrier layer comprising an outward-facing surface of the gusset assembly; and
an edge barrier that overlies portions of the wearer-facing surface proximate the perimeter edge within the crotch portion and effectively continues over and around the perimeter edge from the wearer-facing surface to the outward-facing surface in the crotch region, wherein the edge barrier comprises one or more section(s) of liquid impermeable edge sealing strip material joined to the barrier layer proximate the perimeter edge, or, wherein the edge barrier comprises an extension of the barrier layer;
wherein the gusset assembly is affixed within the crotch portion by a pattern of discrete deposits of adhesive.

2. The durable absorbent pant of claim 1, wherein the deposits of adhesive in the pattern have an average size of 0.25 mm 2 to 2.0 mm².

3. The durable absorbent pant of claim 1, wherein an area of an outward-facing surface of the gusset assembly, entirely surrounded by but longitudinally and laterally inboard of the perimeter edge, is not affixed to the crotch portion.

4. The durable absorbent pant of claim 1, wherein the deposits of adhesive in the pattern have the form of substantially circular dots.

5. The durable absorbent pant of claim 1, wherein the pattern of deposits of adhesive follow paths that generally parallel the paths of the leg opening edges and are disposed inboard thereof and proximate thereto.

6. The durable absorbent pant of claim 1, wherein the pattern of deposits of adhesive reflects programmed contours conforming to the paths of the leg opening edges.

7. The durable absorbent pant of claim 1, wherein the deposits of adhesive affix one or both the barrier layer and edge barrier directly to the crotch portion.

8. A durable absorbent pant, comprising:
an outer pant structure comprising:
a front waist portion with a front waist edge;
a rear waist portion with a rear waist edge;
a crotch portion having a forward portion meeting the front waist portion and a rearward portion meeting the rear waist portion; and
left and right hip side portions at which the front waist portion meets the rear waist portion to form a waist opening with a waist opening edge comprising the front waist edge and the rear waist edge, and to form left and right leg openings with left and right leg opening edges; and
a gusset assembly disposed at least partially in the crotch portion, the gusset assembly having a wearer-facing surface, an outward-facing surface and a perimeter edge, and comprising:
an absorbent layer;
a liquid-impermeable barrier layer disposed beneath the absorbent layer, the barrier layer comprising an outward-facing surface of the gusset assembly; and
an edge barrier that overlies portions of the wearer-facing surface proximate the perimeter edge within the crotch portion and effectively continues over and around the perimeter edge from the wearer-facing surface to the outward-facing surface in the crotch region, wherein the edge barrier comprises one or more section(s) of liquid impermeable edge sealing strip material joined to the barrier layer proximate the perimeter edge, or, wherein the edge barrier comprises an extension of the barrier layer;
an extending flange comprising a fabric material having a width, the entirety of the width being bonded to the edge barrier, the flange extending laterally outboard beyond a proximate side edge of the absorbent layer in the crotch portion, the extending flange being affixed to the outer pant structure.

9. The durable absorbent pant of claim 8, wherein the gusset assembly comprises an elastic stretch knit fabric.

10. The durable absorbent pant of claim 8, wherein the barrier layer comprises an elastomeric film.

11. The durable absorbent pant of claim 8, wherein the edge barrier comprises an elastomeric film.

12. The durable absorbent pant of claim 8, comprising a supplemental elastic stretch knit fabric trip strip having a first strip portion affixed to the gusset assembly at a first attachment location and a second strip portion affixed to the front or rear waist portion at a second attachment location, wherein the deposits of adhesive are present at either or both the first attachment location and second attachment location.

13. A durable absorbent pant, comprising:
an outer pant structure comprising:
a front waist portion with a front waist edge;
a rear waist portion with a rear waist edge;
a crotch portion having a forward portion meeting the front waist portion and a rearward portion meeting the rear waist portion; and
left and right hip side portions at which the front waist portion meets the rear waist portion to form a waist opening with a waist opening edge comprising the front waist edge and the rear waist edge, and to form left and right leg openings with left and right leg opening edges; and
a gusset assembly disposed at least partially in the crotch portion, the gusset assembly having a wearer-facing surface, an outward-facing surface and a perimeter edge having forward and rearward edge portions respectively in the front waist portion and rear waist portions, and left and right crotch edge portions, and comprising:
an absorbent layer;
a liquid-impermeable barrier layer disposed beneath the absorbent layer, the barrier layer comprising an outward-facing surface of the gusset assembly; and
an edge barrier that overlies portions of the wearer-facing surface proximate the perimeter edge within the crotch portion and effectively continues over and around the perimeter edge from the wearer-facing surface to the outward-facing surface in the crotch region, wherein the edge barrier comprises one or more section(s) of liquid impermeable edge sealing strip material joined to the barrier layer proximate the perimeter edge, or, wherein the edge barrier comprises an extension of the barrier layer;
wherein the gusset assembly is affixed to the outer pant structure in the front and rear waist portions, respectively, and is unaffixed to the outer pant structure along portions proximate the leg opening edges.

14. The absorbent pant of claim 13, wherein:
the gusset assembly is affixed to the outer pant structure at forward and rearward seams
the gusset assembly has a first length between the seams, when in a relaxed condition;
the outer pant structure has a second length between the seams, when in a relaxed condition; and
the first length is equal to or greater than the second length.

15. The absorbent pant of claim 13, wherein:
the gusset assembly is affixed to the outer pant structure at forward and rearward seams
the gusset assembly has a first length between the seams, when in a relaxed condition;
the outer pant structure has a second length between the seams, when in a relaxed condition; and
the first length is less than the second length.
